(19) United States
(12) Patent Application Publication
(10) Pub. No.: US 2025/0045844 A1
(43) Pub. Date: Feb. 6, 2025

(54) ENVIRONMENTAL ATTRIBUTES FOR COSMETIC INGREDIENTS

(71) Applicant: BASF SE, Ludwigshafen (DE)

(72) Inventors: Bastian Grumbrecht, Ludwigshafen (DE); Steffen Thomas Klosterhalfen, Ludwigshafen (DE); Christian Krueger, Ludwigshafen (DE); Kurt Wenzl, Ludwigshafen (DE); Ana Alba Perez, Ludwigshafen (DE); Christopher Alec Anderlohr, Ludwigshafen (DE); Martin Binder, Ludwigshafen (DE); Alessandro Pistillo, Ludwigshafen (DE)

(21) Appl. No.: 18/720,099

(22) PCT Filed: Dec. 19, 2022
(86) PCT No.: PCT/EP2022/086607

§ 371 Date: Jun. 14, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06Q 50/04

(52) U.S. Cl. CPC G06Q 50/04

(57) ABSTRACT

Disclosed are systems for producing a cosmetic ingredient associated with a digital asset, methods for producing a cosmetic ingredient associated with a digital asset, apparatuses for generating a digital asset, computer-implemented methods for generating a chemical passport, computer program elements for generating a digital asset, uses of a cosmetic ingredient associated with a digital asset, uses of a digital asset, products produced from the cosmetic ingredient and associated with a digital asset, a digital asset including one or more decentral identifier(s) and data related to the environmental impact data, apparatuses for producing a product associated with the digital asset and methods for producing a product associated with the digital asset.

### TECHNICAL FIELD

The present disclosure relates to systems for producing a cosmetic ingredient associated with a cosmetic ingredient passport or digital asset, methods for producing a cosmetic ingredient associated with a cosmetic ingredient passport or digital asset, apparatuses for generating a passport or digital asset associated with a cosmetic ingredient, computer-implemented methods for generating a cosmetic ingredient passport or digital asset, computer program elements for generating a cosmetic ingredient passport or digital asset, uses of a cosmetic ingredient associated with a cosmetic ingredient passport or digital asset, uses of a cosmetic ingredient passport or digital asset, products produced from the cosmetic ingredient and associated with a cosmetic ingredient passport or digital asset, a cosmetic ingredient, a cosmetic ingredient passport or digital asset including one or more decentral identifier(s) and data related to environmental impact data, apparatuses for producing a product associated with the cosmetic ingredient passport or digital asset and methods for producing a product associated with the cosmetic ingredient passport or digital asset.

### TECHNICAL BACKGROUND

In supply chains the environmental impact of each supply chain participants is of great interest.

Specifically in the field of chemistry, cosmetic ingredients are employed for a wide range of applications and are supplied to diverse value chains. In such complex systems transparency between value chain participants is hard to achieve.

### SUMMARY OF THE INVENTION

In one aspect disclosed is a system for producing a cosmetic ingredient associated with a cosmetic ingredient passport or a digital asset, the system comprising:
a chemical production network configured to produce the cosmetic ingredient wherein the cosmetic ingredient is produced from one or more input material(s) through one or more chemical process(s) of the chemical production network, wherein the one or more input material(s) and/or the one or more chemical process(s) are associated with environmental attribute(s);
a production operating apparatus configured to generate the cosmetic ingredient passport or digital asset by providing a decentral identifier associated with the produced cosmetic ingredient and the one or more environmental attribute(s) of the one or more input material(s) and/or the one or more chemical process(s);
   linking the decentral identifier and the environmental attribute(s);
a chemical production network or system configured to provide the produced cosmetic ingredient in association with the cosmetic ingredient passport or digital asset.

In another aspect disclosed is a system for producing a cosmetic ingredient associated with a cosmetic ingredient passport or digital asset, the system comprising:
a chemical production network configured to produce the cosmetic ingredient from one or more input material(s) through chemical process(s), wherein the one or more input material(s) and/or the chemical process(s) are associated with environmental attribute(s);
a production operating apparatus configured to generate the cosmetic ingredient passport or digital asset by providing and/or linking a decentral identifier associated with the cosmetic ingredient and one or more environmental attribute(s) associated with the one or more input material(s) and/or the chemical process(s).

In another aspect disclosed is a method for producing a cosmetic ingredient associated with a cosmetic ingredient passport or digital asset, wherein the method comprises:
producing the cosmetic ingredient from one or more input material(s) through one or more chemical process(s) of a chemical production network, wherein the one or more input material(s) and/or the one or more chemical process(s) are associated with environmental attribute(s);
generating the cosmetic ingredient passport or digital asset by providing a decentral identifier associated with the produced cosmetic ingredient and one or more environmental attribute(s) associated with the one or more input material(s) and/or the one or more chemical process(s);
   linking the decentral identifier and the one or more environmental attribute(s);
providing the produced cosmetic ingredient in association with the cosmetic ingredient passport or digital asset.

In another aspect disclosed is a method for producing a cosmetic ingredient associated with a cosmetic ingredient passport or a digital asset, wherein the method comprises:
producing the cosmetic ingredient from one or more input material(s) through one or more chemical process(s) of a chemical production network, wherein the one or more input material(s) and/or the one or more chemical process(s) are associated with environmental attribute(s),
generating the cosmetic ingredient passport or digital asset by providing and/or linking a decentral identifier associated with the cosmetic ingredient and one or more environmental attribute(s) of the one or more input material(s) and/or the chemical process(s).

In another aspect disclosed is an apparatus for generating a passport or digital asset associated with a cosmetic ingredient, wherein the cosmetic ingredient is produced from one or more input material(s) through one or more chemical process(s) of a chemical production network, wherein the one or more input material(s) and/or the one or more chemical process(s) are associated with environmental attribute(s), the apparatus comprising:
a decentral identity provider configured to provide a decentral identifier associated with the produced cosmetic ingredient and one or more environmental attribute(s) of the one or more input material(s) and/or the one or more chemical process(s) used to produce the cosmetic ingredient;
an assignor configured the decentral identifier and the environmental attribute(s);
a passport provider configured to provide the cosmetic ingredient passport or digital asset in association with the produced cosmetic ingredient to a decentral network, wherein the environmental attribute(s) associated with the cosmetic ingredient is made accessible to a consumer of the cosmetic ingredient through the cosmetic ingredient passport or digital asset.

In another aspect disclosed is a method, e.g. a computer-implemented method, for generating a cosmetic ingredient passport or digital asset associated with a cosmetic ingredient, wherein the cosmetic ingredient is produced from one or more input material(s) through one or more chemical process(s) of a chemical production network, wherein the one or more input material(s) and/or the one or more chemical process(s) are associated with environmental attribute(s), the method comprising:
providing a decentral identifier associated with the produced cosmetic ingredient and one or more environmental attribute(s) of the one or more input material(s) and/or the one or more chemical process(s) used to produce the cosmetic ingredient;
linking the decentral identifier and the environmental attribute(s);
providing the cosmetic ingredient passport or digital asset in association with the produced cosmetic ingredient to a decentral network, wherein the environmental attribute(s) associated with the cosmetic ingredient is made accessible to a user of the cosmetic ingredient through the cosmetic ingredient passport or digital asset.

In another aspect disclosed is a computer element, such as a computer readable storage medium, a computer program or a computer program product, comprising instructions, which when executed by a computing node or a computing system, direct the computing node or computing system to carry out the steps of the computer-implemented methods disclosed herein.

In another aspect disclosed is a computer element, such as a computer readable storage medium, a computer program or a computer program product, comprising instructions, which when executed by the apparatuses disclosed herein, direct the apparatuses to carry out steps the apparatuses disclosed herein are configured to execute.

In another aspect disclosed is a cosmetic ingredient associated with a cosmetic ingredient passport or a digital asset as produced according to the methods disclosed herein. In another aspect disclosed is a cosmetic ingredient associated with a cosmetic ingredient passport or digital asset as produced according to the systems disclosed herein.

In another aspect disclosed is a cosmetic ingredient associated with a cosmetic ingredient passport or digital asset, wherein the cosmetic ingredient is produced from one or more input material(s) through one or more chemical process(s) of a chemical production network, wherein the one or more input material(s) and/or the one or more chemical process(s) are associated with environmental attribute(s), wherein the cosmetic ingredient passport or digital asset includes a decentral identifier associated with the produced cosmetic ingredient and a link to environmental attribute(s) associated with one or more environmental attribute(s) of one or more input material(s) and/or one or more chemical process(s) used to produce the cosmetic ingredient.

In another aspect disclosed is a cosmetic ingredient passport or digital asset as generated according to the methods disclosed herein. In another aspect disclosed is a cosmetic ingredient passport or digital asset as generated according to the apparatuses disclosed herein.

In another aspect disclosed is a production system for producing a product from the cosmetic ingredient associated with the cosmetic ingredient passport or digital asset as provided according to the systems, apparatuses or methods disclosed herein. In another aspect disclosed is a production method for producing a product from the cosmetic ingredient associated with the cosmetic ingredient passport or digital asset as provided according to the systems, apparatuses or methods disclosed herein.

In another aspect disclosed is a use of the cosmetic ingredient associated with the cosmetic ingredient passport or digital asset as disclosed herein for producing a product from the cosmetic ingredient associated with the cosmetic ingredient passport or digital asset.

In another aspect disclosed is a use of the cosmetic ingredient passport or digital asset as disclosed herein for generating a product passport or digital asset associated with a product produced from the cosmetic ingredient associated with the cosmetic ingredient passport or digital asset. In another aspect disclosed is a method for using the digital asset generated according to the methods disclosed herein in the production of a product produced from the cosmetic ingredient associated with the cosmetic ingredient passport or digital asset.

In another aspect disclosed is a cosmetic ingredient associated with a digital asset including a decentral identifier associated with the cosmetic ingredient and linked to one or more environmental attribute(s) of the one or more input material(s) and/or the one or more chemical process(s) used to produce the cosmetic ingredient.

In another aspect disclosed is a use of the cosmetic ingredient associated with the cosmetic ingredient passport or digital asset for producing a product from the cosmetic ingredient and associating the cosmetic ingredient passport or digital asset with the product produced from the cosmetic ingredient. In another aspect disclosed is a use of the cosmetic ingredient associated with the cosmetic ingredient passport or digital asset for producing a product from the cosmetic ingredient and deriving a product passport or digital asset from the cosmetic ingredient passport or digital asset. In another aspect disclosed is a method for using the cosmetic ingredient associated with the digital asset for producing a product from the cosmetic ingredient as disclosed herein and deriving a digital asset associated with the product from the cosmetic ingredient passport or digital asset.

Any disclosure and embodiments described herein relate to the methods, the systems, chemical products, cosmetic ingredient, cosmetic ingredient passports or digital assets and the computer elements lined out above and below and vice versa. Advantageously, the benefits provided by any of the embodiments and examples equally apply to all other embodiments and examples.

### EMBODIMENTS

In the following, embodiments of the present disclosure will be outlined byways of embodiments and/or example. It is to be understood that the present disclosure is not limited to said embodiments and/or examples. All terms and definitions used herein are understood broadly and have their general meaning.

The methods, the systems, cosmetic ingredients, cosmetic ingredient passports or digital assets and the computer elements disclosed herein provide an efficient, secure and robust way for sharing or exchanging environmental impact data across different participant nodes in value chains. In particular, by providing cosmetic ingredient specific data via the cosmetic ingredient passport or digital asset, environmental impacts can be shared and made transparent from the cosmetic ingredient to the product produced from such cosmetic ingredient. The cosmetic ingredient passport or digital asset enables secure data exchange, since data access can be controlled by the cosmetic ingredient provider, e.g. the entity providing the cosmetic ingredient. The exchanged data assets can be specific to the cosmetic ingredient as produced and tailored to the needs of the consumer of the cosmetic ingredient. This way an improved tracking and tracing of cosmetic ingredients can be achieved by securely providing environmental impact data in diverse and highly complex value chains. The environmental impact of cosmetic ingredient can hence be tracked leading to simpler, more efficient and sustainable handling of cosmetic ingredient by value chain participants.

The cosmetic ingredient according to the present invention can be any cosmetically acceptable ingredient. These ingredients are known to the person skilled in the art and can be found in several publications, e. g. in the latest edition of the “International Cosmetic Ingredient Dictionary and Handbook” published by the Personal Care Products Council. Another well-known source of cosmetically acceptable ingredients is the cometic ingredient database Cosing. Cosing can be accessed on the internet pages of the European Commission.

### Emollient

The cosmetic ingredient according to the present invention can be an emollient.

In the context of the present invention, the term “emollient” is understood to mean substances that make the skin soft and supple, especially by supplying the skin with lipids or reducing evaporation or increasing the moisture content of the skin. Suitable emollients are substances from the group of the oils, fats, waxes, hydrocarbons and/or organosilicon compounds that are liquid at room temperature or have a melting point <45° C.

Emollients can be oils, fats and/or waxes, for example from the group formed by esters, wax esters, waxes, triglycerides or partial glycerides, natural vegetable oils or fats, hydrocarbons, organosilicon compounds, Guerbet alcohols, mono-/dialkyl ethers, mono-/dialkyl carbonates, and mixtures thereof.

From the group of the esters, examples of esters include those of linear fatty acids with linear or branched fatty alcohols, esters of linear fatty alcohols with linear or branched carboxylic acids, esters of alkylhydroxycarboxylic acids with linear or branched fatty alcohols, esters of linear or branched fatty acids with polyhydric alcohols such as diols or trimer triol, wax esters, triglycerides or partial glycerides (called mono-/di-/triglyceride esters), esters of fatty alcohols and/or Guerbet alcohols with aromatic carboxylic acids, esters of dicarboxylic acids with linear or branched alcohols, natural vegetable oils or fats and mixtures thereof.

Suitable examples from the group of esters of linear C₆-C₂₂ fatty acids with linear or branched C₆-C₂₂ fatty alcohols or esters of branched C₆-C₂₂ carboxylic acids with linear or branched C₆-C₂₂ fatty alcohols are myristyl myristate (Cetiol® MM), myristyl isostearate, myristyl oleate, myristyl erucate, cetyl isostearate, cetyl oleate, cetyl erucate, stearyl myristate, stearyl isostearate, stearyl oleate, stearyl erucate, isostearyl myristate, isostearyl palmitate, isostearyl stearate, isostearyl isostearate, isostearyl oleate, Isopropyl Myristate, Isopropyl Palmitate, oleyl myristate, oleyl palmitate, oleyl stearate, oleyl isostearate, oleyl oleate, oleyl behenate, oleyl erucate (Cetiol® J 600), behenyl oleate, behenyl erucate, erucyl myristate, erucyl palmitate, erucyl stearate, erucyl isostearate, erucyl oleate, erucyl behenate and erucyl erucate, Ethylhexyl Stearate (Cetiol® 868), Hexyl Laurate (Cetiol® A), Coco-Caprylate (Cetiol® C5), Coco-Capry-late/Caprate (Cetiol® LC, Cetiol® C 5C), Propylheptyl Caprylate (Cetiol® Sensoft), Cetearyl Isononanoate (Cetiol® SN), Decyl Oleate (Cetiol® V), Cetearyl Ethylhexanoate.

Additionally suitable are esters of alkylhydroxycarboxylic acids with linear or branched C₆-C₂₂ fatty alcohols, preferably esters of lactic acid such as lauryl lactate.

Additionally suitable are esters of dicarboxylic acids and linear or branched alcohols, preferably esters of malic acid, adipic acid and/or sebacic acid, such as dibutyl adipate, dioctyl malate and/or diisopropyl sebacate.

Also suitable are esters of linear and/or branched fatty acids with polyhydric alcohols (such as propylene glycol, dimer diol or trimer triol), such as Propylene Glycol Dicaprylate/Dicaprate (Myritol® PGDC), triglycerides based on C₆-C₁₀ fatty acids, liquid mono-/di-/triglyceride mixtures based on C₆-C₁₈ fatty acids (Myritol® 331, Myritol® 312, Myritol® 318), esters of C₆-C₂₂ fatty alcohols and/or Guerbet alcohols with aromatic carboxylic acids, especially benzoic acid, esters of benzoic acid with linear and/or branched C₆-C₂₂ alcohols (e.g. Finsolv® TN, Cetiol® AB).

Of good suitability as natural, especially vegetable, fats and oils are groundnut oil, soybean oil, jojoba oil, rapeseed oil, avocado oil, argan oil, castor oil, sunflower oil, palm oil, palm kernel oil, linseed oil, almond oil, wheat germ oil, macadamia nut oil, olive oil, sesame oil, cocoa butter and shea butter, for example Cegesoft® PFO, Cegesoft® PS 6, Cegesoft® SBE, Cegesoft® SH, Cegesoft® VP or Cetiol® SB 45.

Also usable as emollient are, for example, natural vegetable waxes such as fruit waxes (for example orange waxes) and animal waxes such as wool wax.

Also suitable as emollient are C₁₂-C₁₅ fatty alcohols that are usually obtained from natural fats, oils and waxes, such as lauryl alcohol, myristyl alcohol or 1-pentadecanol.

Further suitable emollients are organosilicon compounds, which are frequently referred to simply as silicones. They may take the form of cyclic, branched or linear silicones. Silicones are high molecular weight synthetic polymeric compounds in which silicon atoms are joined via oxygen atoms in a chain-like and/or grid-like manner and the remaining valences of silicon are satisfied by hydrocarbon radicals (usually methyl, more rarely ethyl, propyl, phenyl groups etc.). Systematically, the silicones are referred to as polyorganosiloxanes.

Advantageous polyorganosiloxanes are, for example, the methyl-substituted polyorganosiloxanes that can be represented by the following structural formula:

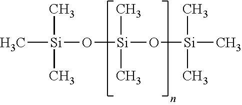

They are also referred to as Polydimethylsiloxane or Dimethicone (INCI). Dimethicones come in various chain lengths and with various molecular weights. They are available, for example, under the Abil® 350 trade name from Evonik or Xiameter PMX-200 Silicone Fluid trade name from Dow Chemicals.

Also advantageous are phenylmethylpolysiloxane (INCI: Phenyl Dimethicone, Phenyl Trimethi-cone), cyclic silicones (e.g. decamethylcyclopentasiloxane or dodecamethylcyclohexasiloxane), which are also referred to in accordance with INCI as Cyclomethicone, amino-modified silicones (INCI: Amodimethicone) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Evonik.

Silicones which are particularly preferred in accordance with the invention are Dimethicone and Cyclomethicone.

Further suitable emollients are mono- and/or dialkyl carbonates of linear or branched C₆-C₂₂ fatty alcohols, such as Dicaprylyl Carbonate (Cetiol® CC) or dipropylheptyl carbonate (Cetiol® 4 All), Guerbet carbonates based on fatty alcohols having 6 to 18, preferably 8 to 10, carbon atoms, linear or branched, symmetric or unsymmetric dialkyl ethers having 6 to 22 carbon atoms per alkyl group, for example Dicaprylyl Ether (Cetiol® OE). Further suitable emollients are hydrocarbons such as mineral oils, Paraffinum Liquidum, Undecane/Tridecane (Cetiol® Ultimate), Hydrogenated Polyisobutene (Luvitol® Lite), substituted cyclohexanes, isoparaffins or paraffins.

Suitable Guerbet alcohols are those based on fatty alcohols having 6 to 18, preferably 8 to 10, carbon atoms (Eutanol® G, Eutanol® G 16).

Further examples of emollients are dibutyl adipate, dioctyl carbonate, dipropylheptyl carbonate, octyldodecanol, undecane/tridecane, Caprylic/Capric Triglyceride, C₁₂-C₁₅-alkyl benzoate, ethylhexyl stearate, myristyl myristate, glyceryl oleate, isopropyl palmitate, dioctyl ether, octadecenyl docosenoate, rapeseed oil, wheatgerm oil, and Olus Oil.

### Surfactant

The cosmetic ingredient according to the present invention can be a surfactant, hereinafter also referred to as interface-active substances.

In the context of the present invention, the term “interface-active substances” is understood to mean compounds that lower the interfacial tension of a liquid or interfacial tension between two phases. Such compounds are also referred to as surfactants or emulsifiers.

The surfactant can be nonionic, anionic, cationic and/or amphoteric or zwitterionic.

Examples of suitable nonionic interface-active substances are
(1) Addition products of 2 to 50 mol of ethylene oxide and/or 1 to 20 mol of propylene oxide onto linear fatty alcohols having 8 to 40 carbon atoms, onto fatty acids having 12 to 40 carbon atoms and onto alkylphenols having 8 to 15 carbon atoms in the alkyl group
(2) C₁₂-C₁₈ fatty acid mono- and diesters of addition products of 1 to 50 mol of ethylene oxide onto glycerol
(3) Sorbitan mono- and diesters of saturated and unsaturated fatty acids having 6 to 22 carbon atoms and the ethylene oxide addition products thereof
(4) Alkyl mono- and oligoglycosides, preferably having 8 to 22 carbon atoms in the alkyl radical, and ethoxylated analogs thereof
(5) Addition products of ethylene oxide onto castor oil and/or hydrogenated castor oil, for example with 2 to 15 mol or with 40 to 60 mol of ethylene oxide
(6) Polyol and especially polyglyceryl esters, such as polyol poly-12-hydroxystearates, polyglyceryl polyhydroxystearate, polyglyceryl polyricinoleate, polyglyceryl diisostearate or polyglyceryl dimerate. Likewise suitable are mixtures of compounds of two or more of these substance classes
(7) Lecithins and phospholipids
(8) Partial esters based on linear, branched, unsaturated or saturated C₆-C₂₂ fatty acids, ric-inoleic acid and 12-hydroxystearic acid and polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose), or mixed esters, for example glyceryl stearate citrate and glyceryl stearate lactate.
(9) Polysiloxane-polyalkyl-polyether copolymers and corresponding derivatives (called silicone emulsifiers)
(10) Mixed esters of pentaerythritol, fatty acids, citric acid and fatty alcohol and/or mixed esters of fatty acids having 6 to 22 carbon atoms, methylglucose and polyols, preferably glycerol or polyglycerol, and/or
(11) Mixed esters of fatty acids having 6 to 22 carbon atoms with sucrose

The addition products of ethylene oxide and/or of propylene oxide onto fatty alcohols, fatty acids, alkylphenols, glycerol mono- and diesters, and also sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. According to the degree of ethoxylation, the interface-active compounds can emulsify water in oil or oil in water. C_{12/18} fatty acid mono- and diesters of addition products of ethylene oxide onto glycerol are also known as refatting agents in cosmetic formulations.

Examples of suitable commercially available interface-active substances of the nonionic type are Cetyl Dimethicone Copolyol (e.g. Abil EM-90), Polyglyceryl-2 Dipolyhydroxystearate (e.g. Dehymuls PGPH), Polyglyceryl-3 Diisostearate (e.g. Lameform TGI), Polyglyceryl-4 Isostearate (e.g. Isolan GI 34), Polyglyceryl-3 Oleate (e.g. Isolan GO 33), Diisostearoyl Polyglyceryl-3 Diisostearate (e.g. Isolan PDI), Polyglyceryl-3 Methylglucose Distearate (e.g. Tego Care 450), Polyglyceryl-3 Beeswax (e.g. Cera Bellina), Polyglyceryl-4 Caprate (e.g. Polyglycerol Caprate T2010/90), Polyglyceryl-3 Cetyl Ether (e.g. Chimexane NL), Polyglyceryl-3 Distearate (e.g. Cremophor GS 32) and Polyglyceryl Polyricinoleate (e.g. Admul WOL 1403), Glyceryl Oleate (e.g. Monomuls 90-0 18), Alkyl Glucoside (e.g. Plantacare 1200, Emulgade PL 68/50, Montanov 68, Tego Care CG 90, Tego Glucosid L 55), Methyl Glucose Isostearate (e.g. Tego Care IS), Methyl Glucose Sesquistearate (Tego Care PS), Sucrose Ester (e.g. Crodesta F-10, F-20, F-50, F-70, F-110, F-160, SL-40, Emulgade® Sucro), ethoxylated and/or propoxylated fatty alcohols, fatty acids, castor oils and hydrogenated castor oils (e.g. Eumulgin® B1, B2, B3, BA 25, L, CO 40, CO 60, 0 10, 0 30, S 2, S 20, Cremophor® WO 7, Arlacel 989), PEG-30 Dipolyhydroxystearate (e.g. Arlacel P 135, Dehymuls LE), sorbitan esters, sorbitan esters ethoxylated and/or propoxylated, and mixtures thereof. A particularly effective mixture consists of Polyglyceryl-2 Dipolyhydroxystearate and Lauryl Glucoside and glycerol (e.g. Eumulgin VL 75). Also suitable are Polyglyceryl-4 Diisostearate/Polyhydroxystearate/Sebacate (Isolan GPS) or Diisostearoyl Polyglyceryl-3 Diisostearate (e.g. Isolan PDI).

Further nonionic interface-active substances are reaction products of poly-12-hydroxystearic acid with polyglycerols of the following homolog distribution (preferred amounts are given in brackets):
Glycerols: 5 to 35 (15 to 30) % by weight
Diglycerols: 15 to 40 (20 to 32) % by weight
Triglycerols: 10 to 35 (15 to 25) % by weight
Tetraglycerols: 5 to 20 (8 to 15) % by weight
Pentaglycerols: 2 to 10 (3 to 8) % by weight
Oligoglycerols: ad 100% by weight

In one embodiment of the invention, the glyceryl ester is the diester of polyhydroxystearic acid, Polyglyceryl-2 Dipolyhydroxystearate, which is sold, for example, by BASF Personal Care and Nutrition GmbH under the Dehymuls® PGPH name on its own or blends thereof under the Eumulgin® VL 75 name (blend with Lauryl Glucosides in a weight ratio of 1:1, O/W emulsifier) or Dehymuls® SBL name (W/O emulsifier) by BASF Personal Care and Nutrition Deutschland GmbH.

Nonionic interface-active compounds from the group of the alkyl mono- or alkyl oligoglycosides are particularly skin-friendly. C₈-C₂₂-Alkyl mono- and oligoglycosides are known from the prior art. They are prepared especially by reacting glucose or oligosaccharides with primary alcohols having 8 to 22 carbon atoms, preferably 12 to 22, and more preferably 12 to 18 carbon atoms. As regards the glycoside radical, either monoglycosides, in which a cyclic sugar radical is glycosidically bonded to the fatty alcohol, or oligomeric glycosides with a degree of oligomerization up to preferably about 8 are suitable. The degree of oligomerization here is a statistical average value which is based on a homolog distribution customary for such technical-grade products. Products available under the Plantacare® name comprise a glucosidically bonded C₈-C₁₆-alkyl group onto an oligoglucoside radical having an average degree of oligomerization of 1 to 2. The acylglucamides derived from glucamine are also suitable.

Also suitable as nonionic interface-active compounds are substances such as lecithins and phospholipids. Examples of natural lecithins include the cephalins, which are also referred to as phosphatidic acids and are derivatives of 1,2-diacyl-sn-glycerol-3-phosphoric acids. In contrast, phospholipids are usually understood to mean mono- and preferably diesters of phosphoric acid with glycerol (glycerol phosphates), which are generally counted among the fats. In addition, sphingosines or sphingolipids are also possible.

Examples of nonionic interface-active compounds that may be present include silicone emulsifiers. These may be selected, for example, from the group of alkylmethicone copolyols and/or alkyldimethicone copolyols, especially from the group of compounds which are characterized by the following chemical structure:

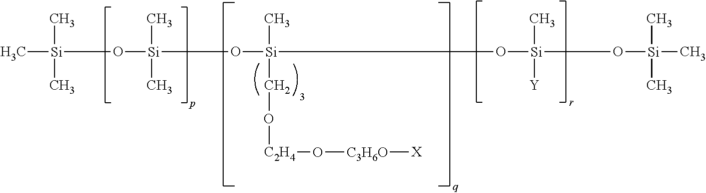

in which X and Y are independently selected from the group of H (hydrogen) and the branched and unbranched alkyl groups, acyl groups and alkoxy groups having 1-24 carbon atoms, p is 0-200, q is 1-40, and r is 1-100.

One example of silicone emulsifiers is that of dimethicone copolyols, which are sold by Evonik under the AXIL® B 8842, ABIL® B 8843, ABIL® B 8847, ABIL® B 8851, ABIL® B 8852, ABIL®B 8863, ABIL® B 8873 and ABIL®B 88183 trade names. A further example is Cetyl PEG/PPG-10/1 Dimethicone (Cetyl Dimethiconecopolyol), which is sold by Evonik under the ABIL® EM 90 brand name. A further example is Cyclomethicone Dimethiconecopolyol, which is sold by Evonik under the ABIL® EM 97 and ABIL® WE 09 brand name. Also suitable are Lauryl PEG/PPG-18/18 Methicone (Laurylmethiconecopolyol), available under the Dow Corning® 5200 Formulation Aid brand-name from the company Dow Corning Ltd., and Octyl Dimethicon Ethoxy Glucosid from Wacker.

Also suitable as interface-active compounds are zwitterionic, ampholytic and/or cationic compounds.

Interface-active compounds which bear at least one quaternary ammonium group and at least one —COO(−)— or —SO3(−)— group in the molecule are referred to as as zwitterionic.

Particularly suitable zwitterionic compounds are the so-called betaines, such as the N-alkyl-N,N-dimethylammonium glycinates, for example cocoalkyldimethylammonium glycinate, N-ac-ylaminopropyl-N,N-dimethylammonium glycinates, for example cocoacylaminopropyldime-thylammonium glycinate, and 2-alkyl-3-carboxylmethyl-3-hydroxyethylimidazoline having in each case 8 to 18 carbon atoms in the alkyl or acyl group, and also cocoacylaminoethyl hydroxyethylcarboxymethylglycinate. A preferred zwitterionic surface-active compound is the fatty acid amide derivative known by the INCI name Cocamidopropyl Betaine.

Interface-active compounds which, aside from a C₈-C₁₈-alkyl or acyl group, comprise at least one free amino group and at least one —COOH or —SO₃H group in the molecule and are capable of forming internal salts are called ampholytic. Examples of suitable ampholytic compounds are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkyla-minopropionic acids and alkylaminoacetic acids each having about 8 to 18 carbon atoms in the alkyl group.

Usable cationic interface-active compounds are especially quaternary ammonium compounds. Preference is given to ammonium halides, in particular chlorides and bromides, such as alkyltrimethylammonium chlorides, dialkyldimethylammonium chlorides and trialkylmethylammonium chlorides, e.g. cetyltrimethylammonium chloride, hexadecyltrimethylammonium chloride, stearyltrimethylammonium chloride, distearyldimethylammonium chloride, lauryldimethylben-zylammonium chloride and tricetylmethylammonium chloride. Additionally suitable are fatty amines such as Stearamidopropyl Dimethylamine (Dehyquart® S18) and the very readily biodegradable quaternary ester compounds, for example the dialkylammonium methosulfates and methylhydroxyalkyldialkoyloxyalkylammonium methosulfates sold under the trade name Stepantex® and the corresponding products of the Dehyquart® series, be used as cationic interface-active compound. The term “ester quats” is generally understood to mean quaternized fatty acid triethanolamine ester salts. Likewise suitable are the quaternized protein hydrolyzates.

Also suitable are interface-active compounds having at least one anionic group such as a carboxylate, sulfate, sulfonate or phosphate group. Examples of suitable interface-active compounds, each in the form of their salts, are fatty acids, ether carboxylic acids, acyl sarcosides having 8 to 24 carbon atoms in the acyl group, acyl taurides having 8 to 24 carbon atoms in the acyl group, acyl isethionates having 8 to 24 carbon atoms in the acyl group, acyl glutamates having 8 to 24 carbon atoms in the acyl group, sulfosuccinic mono- and dialkyl esters having 8 to 24 carbon atoms in the alkyl group and sulfosuccinic monoalkyl polyoxyethyl esters having 8 to 24 carbon atoms in the alkyl group and 1 to 6 oxyethyl groups, linear alkanesulfonates having 8 to 24 carbon atoms, alkylarylsulfonates, linear alpha-olefinsulfonates having 8 to 24 carbon atoms, alpha-sulfo fatty acid methyl esters of fatty acids having 8 to 30 carbon atoms, alkyl sulfates, alkyl polyglycol ether sulfates, esters of tartaric acid and esters of citric acid, alkyl and/or alkenyl ether phosphates, sulfated fatty acid alkylene glycol esters, monoglyceride sulfates and monoglyceride ether sulfates and condensation products of C8-C30 fatty alcohols with protein hydrolyzates and/or amino acids and derivatives thereof, called protein fatty acid condensates, e.g. Lamepon®, Gluadin®, Hostapon® KCG or Amisoft.

Typically, the salts are selected from the sodium, potassium and ammonium salts, and the mono-, di- and trialkanolammonium salts having 2 to 4 carbon atoms in the alkanol group.

Suitable examples are sodium acylglutamate, Myristoyl Sarcosine, TEA-Lauroyl Sarcosinate, sodium lauroylsarcosinate and sodium cocoylsarcosinate, sodium or ammonium cocoylisethio-nate, dioctylsodium sulfosuccinate, disodium laureth sulfosuccinate, disodium laurylsulfosuc-cinate and disodium undecylenamido MEA sulfosuccinate, Disodium PEG-5 Laurylcitratesul-fosuccinate, sodium, ammonium, magnesium, MIPA, TIPA laureth sulfate, sodium myrethsul-fate and sodium C₁₂₋₁₃ parethsulfate, sodium C12-15 Pareth-15 Sulfonate, sodium, ammonium and TEA laurylsulfate, sodium lauroyltaurate and sodium methylcocoyltaurate, sodium laureth-13 carboxylate and Sodium PEG-6 Cocamide Carboxylate, Sodium PEG-7-Olive Oil Carboxylate, DEA-Oleth-10 Phosphate and Dilaureth-4 Phosphate, sodium cocomonoglyceridesulfate, Sodium C₁₂₋₁₄ Olefinsulfonate, sodium laurylsulfoacetate, Magnesium PEG-3 Cocamidesulfate, di-TEA-palmitoyl aspartate and Sodium Caprylic/Capric Glutamate, Palmitoyl Hydrolyzed Milk Protein, Sodium Cocoyl Hydrolyzed Soy Protein and Sodium/Potassium Cocoyl Hydrolyzed Collagen, calcium stearoyllactylate, Laureth-6 Citrate and Sodium PEG-4 Lauramide Carboxylate.

Anionic compounds of excellent suitability are alk(en)yl polyglycol ether citrates and especially mixtures of mono-, di- and triesters of citric acid and alkoxylated alcohols that conform to the formula (I):

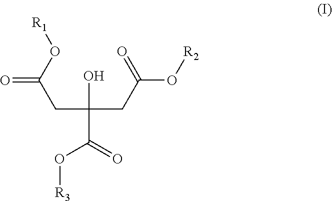

in which
R₁, R₂ and R₃ are independently hydrogen or the radical of the formula

R₄(OCH₂CHR₅)ₙ (II)
in which
R₄ is a linear or branched alkyl and/or alkenyl radical having 6 to 22 carbon atoms,
R₅ is hydrogen or methyl radical and
n is a number from 1 to 20, with the condition that at least one of the R₁, R₂ and R₃ radicals is not hydrogen.

Typical examples of the alcohol moiety of the esters are addition products of an average of 1 to 20 mol, preferably 5 to 10 mol, of ethylene oxide and/or propylene oxide onto caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachiyl alcohol, gadoleyl alcohol, behenyl alcohol, erucyl alcohol and brassidyl alcohol, and technical grade mixtures thereof.

It is particularly preferable in the context of the invention to use, as anionic interface-active substances, salts of alkyl polyglycol ether sulfates, salts of acyl isethionates, salts of acyl glutamate, salts of mono- and dialkyl sulfosuccinates having 8 to 24 carbon atoms in the alkyl group, salts of alkyl sulfates, salts of alkyl phosphates, salts of fatty acids, citric esters of glyceryl stearate, and the alkyl polyalkylene glycol ether citrates that have already been discussed in detail above, especially the alkyl polyalkylene glycol ether citrates that have already been discussed in detail above.

In one embodiment of the present invention interface-active substances are nonionic and/or anionic interface-active compounds. Preferred nonionic interface-active compounds are those from the group formed by polyglyceryl esters, sucrose esters, ethoxylated fatty alcohols and ethoxylated fatty acids and alkyl(oligo)glucosides.

Preferred anionic interface-active compounds are those from the group formed by alkyl polyalkylene glycol ether citrates, salts of acyl glutamate, salts of mono- and dialkyl sulfosuccinates having 12 to 24 carbon atoms in the alkyl group, salts of alkyl sulfates and alkyl phosphates, and citric esters of glyceryl stearate.

A suitable subtype is that of mixtures consisting of two different nonionic interface-active compounds, preferably a mixture of 2 different interface-active compounds selected from the group formed by polyglyceryl esters, ethoxylated fatty alcohols and alkyl (oligo)glucosides.

A further suitable subtype is that of mixtures consisting of at least one anionic interface-active compound and at least one nonionic interface-active compound, preferably of one of the anionic interface-active compounds selected from the group of the alkyl polyalkylene glycol ether citrates, salts of acyl glutamate and salts of mono- and dialkyl sulfosuccinates having 12 to 24 carbon atoms in the alkyl group, and one of the nonionic interface-active compounds selected from the group of the polyglyceryl esters, sucrose esters and alkyl (oligo)glucosides.

In a further embodiment of the present invention, the interface-active substance is one or more cationic and/or zwitterionic or ampholytic interface-active compounds, preferably the cationic and/or zwitterionic compounds already mentioned above, preferably quaternary ammonium compounds, dialkylammonium methosulfates, methylhydroxyalkyldialkoyloxyalkylammonium methosulfates, quaternized fatty acid triethanolamine ester salts, fatty amines and betaines, especially Distearoyl Hydroxyethylmonium Methosulfate, Dicocoylethyl Hydroxyethylmonium Methosulfate, Hexadecyltrimethylammonium chloride and/or Stearamidopropyl Dimethylamine, Cocoamidopropyl Betaine, cetyltrimethylammonium chloride, hexadecyltrimethylammonium chloride, stearyltrimethylammonium chloride, distearyldimethylammonium chloride, lauryldime-thylbenzylammonium chloride, tricetylmethylammonium chloride, and Stearylamidopropyl Dimethylamine. This embodiment is recommended for production of cosmetic formulations for the hair, especially for the production of conditioner formulations.

### Rheology Modifier

The cosmetic ingredient according to the present invention can be a rheology modifier.

In the context of the present invention, the term “rheology modifier” is understood to mean substances that alter the deformation and flow properties of material. This comprises organic or inorganic compounds, usually macromolecules, which alter the intermolecular forces through formation of cohesion (intramolecular) or adhesion (intermolecular) in such a way that the viscosity of the coherent phase of the cosmetic formulation is preferably increased.

The rheology modifiers are preferably selected from the group formed by anionic, nonionic, cationic and/or zwitterionic polymers, inorganic substances selected from the group of the fumed silicas, bentonites and hectorites, where these may optionally be chemically modified, and organic substances from the group of the fats, waxes, alcohols and/or hydrocarbons having a melting point above 45° C.

### Polymers

The rheology modifier according to the present invention can be a polymer.

Suitable rheology modifiers are anionic, nonionic, cationic and/or zwitterionic polymers having a natural or synthetic basis.

Examples of polymers having a natural basis are:
starch and modified starch
quaternized collagen polypeptides such as Lauryldimonium hydroxypropyl hydrolyzed collagen (Lamequat® L/Grunau)
polysaccharides, especially Xanthan Gum, Carrageenan, Gellan Gum, Acacia Gum, Tara Gum, Cellulose Gum, Scleroglucan, Glucomannan, Guar gum, Agar-Agar, Algi-nate, Pectin,
chemically modified polysaccharides such as methyl hydroxyethylcellulose, hydroxy-ethyl- and hydroxypropylcellulose, carboxymethylcellulose
modified guar polymers, such as those of the Jaguar® brands from Solvay
derivatized cellulose ethers.

Examples of anionic polymers suitable as rheology modifier are polymers or copolymers comprising carboxylic acid groups in particular. Suitable carboxylic acid-group-containing polymers are obtainable, for example, by free-radical polymerization of ethylenically unsaturated monomers m1). Monomers used here comprise at least one free-radically polymerizable, ethylenically unsaturated double bond and at least one anionogenic and/or anionic group per molecule. Suitable monomers are monoethylenically unsaturated mono- and dicarboxylic acids having 3 to 25, preferably 3 to 6, carbon atoms, which can also be used in the form of their salts or anhy-drides. Examples thereof are acrylic acid, methacrylic acid, ethacrylic acid, Q-chloroacrylic acid, crotonic acid, maleic acid, maleic anhydride, itaconic acid, citraconic acid, mesaconic acid, glutaconic acid, aconitic acid and fumaric acid. The monomers additionally include the monoesters of monoethylenically unsaturated dicarboxylic acids having 4 to 10, preferably 4 to 6, carbon atoms, for example of maleic acid, such as monomethyl maleate. The monomers also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypro-pylsulfonic acid, styrenesulfonic acid, 1-acrylamido-2-methylpropanesulfonic acid, vinylphosphonic acid and allylphosophonic acid. The monomers also include the salts of the abovementioned acids, in particular the sodium, potassium and ammonium salts, and the salts with the abovementioned amines. The monomers can be used as they are or as mixtures with one another.

The monomer is preferably selected from acrylic acid, methacrylic acid, ethacrylic acid, chloroacrylic acid, crotonic acid, maleic acid, maleic anhydride, fumaric acid, itaconic acid, citraconic acid, mesaconic acid, glutaconic acid, aconitic acid and mixtures thereof, more preferably acrylic acid, methacrylic acid and mixtures therof. Preference is given to copolymers of acrylic acid and/or methacrylic acid that have a molecular weight in the range from 10 000 to 10 000 000 daltons.

Additionally preferred are copolymers of the abovementioned monomers with monomers m2. Suitable monomers m2) are methyl (meth)acrylate, methyl ethacrylate, ethyl (meth)acrylate, ethyl acrylate, tert-butyl (meth)acrylate, tert-butyl ethacrylate, n-octyl (meth)acrylate, 1,1,3,3-tetramethylbutyl(meth)acrylate, ethylhexyl (meth)acrylate, n-nonyl(meth)acrylate, n-decyl(meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate and mixtures thereof.

Suitable monomers m2) are additionally acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide, pi-peridinyl(meth)acrylamide and morpholinyl(meth)acrylamide, N-(n-octyl)(meth)acrylamide, N-(1,1,3,3-tetramethylbutyl)(meth)acrylamide, N-ethylhexyl(meth)acylamide, N-(n-nonyl)(meth}acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acylamide, N-palmityl(meth)acrylamide, N-heptadecyl(meth)acrylamide, N-nonadecyl(meth)acylamide, N-arachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide and N-lauryl(meth)acrylamide.

Anionic polymers preferred as carboxylic-acid-group-containing polymers are, for example, homopolymers and copolymers of acrylic acid and methacrylic acid and salts thereof. These also include crosslinked polymers of acrylic acid, as are available under the INCI name Carbomer. Such crosslinked homopolymers of acrylic acid are commercially available, for example, under the Carbopoll® name from Lubrizol. Preference is also given to hydrophobic modified crosslinked and uncrosslinked polyacrylic copolymers, such as Carbopol® Ultrez 21 from Lubrizol.

Further examples of suitable anionic polymers are copolymers of acrylic acid and acrylamide and salts thereof; sodium salts of polyhydroxycarboxylic acids, polyurethanes and polyureas. Particularly suitable polymers are copolymers of (meth)acrylic acid and polyether acrylates, where the polyether chain is terminated with a C8-C30-alkyl radical. These include, for example, Acrylates/Beheneth-25-Methacrylate copolymers which are available under the Aculyn® name from Dow. Suitable polymers are additionally copolymers of acrylic acid and methacrylic acid with hydrophobic monomers, for example C4-C30-alkyl esters of meth(acrylic acid), C4-C30-alkyl vinyl esters, C4-C30-alkyl vinyl ethers and hyaluronic acid.

Likewise suitable as rheology modifiers are cationic polymers, for example what are called quaternary ammonium compounds with the INCI name Polyquaternium-22, Polyquaternium-37, Polyquaternium-39, Polyquaternium-47 or Polyquaternium-86.

In addition, it is also possible to use nonionic polymers as rheology modifiers, such as polyethylene glycols having an average molar mass of >800 g/mol or hydrophobically modified, ethoxylated urethane polymers (HEUR for short) such as Polyurethane-39.

Inorganic Substances

The rheology modifier according to the present invention can be an inorganic substance.

In addition, inorganic substances that can also function as rheology modifiers are those selected from the group of the fumed silicas, bentonites and hectorites, for example hydrophilic and hydrophobic fumed silicas that are sold under the Aerosil® trade name inter alia, and bentonites and hectorites or hydrophobically modified hectorites and bentonites such as Benzyldimethylstearylammonium Hectorite, Dimethyldioctylammonium Hectorite or Quaternium-18 Hectorite, and also Benzyldimethylstearylammonium Bentonite or Quaternium-18 Bentonite, which are available, for example, under the Bentone®, Claytone®, Tixogel® trade names.

### Organic Substances

The rheology modifier according to the present invention can be an organic substance.

In addition, organic substances that can also function as rheology modifiers are those selected from the group of the fats, waxes, alcohols and or hydrocarbons having a melting point above 45° C. Rheology modifiers may also be wholly or partly wax esters, i.e. compounds having a melting point of 45° C., such as mono-, di- and/or triglycerides, such as the Cutina® MD or Cutina® GMS (glyceryl stearate) products marketed by BASF Personal Care and Nutrition Deutschland GmbH & Co. KG. Usable rheology modifiers are, for example, also natural vegetable waxes such as candelilla wax, carnauba wax, japan wax, rice germ oil wax, sugarcane wax, montan wax, sunflower wax, and animal waxes such as beeswax.

It is also possible to use hydrogenated or hardened waxes such as the mineral waxes, for example ceresin and ozokerite, or the petrochemical waxes, such as petrolatum, paraffin waxes and microwaxes. Usable rheology modifiers also include chemically modified waxes such as montan ester waxes, Sasol waxes and hydrogenated jojoba waxes. Synthetic waxes usable in accordance with the invention include, for example, wax-like polyalkylene waxes and polyethylene glycol waxes.

Further usable waxes are esters of aromatic carboxylic acids, dicarboxylic acids, tricarboxylic acids and hydroxycarboxylic acids (e.g. 12-hydroxystearic acid) and saturated and/or unsaturated, branched and/or unbranched alcohols. Examples of such esters are the C₁₆-C₄₀-alkyl stearates, C₂₀-C₄₀-alkyl stearates (e.g. Kesterwachs K82H), C₂₀-C₄₀-dialkyl esters of dimer acids, C₁₃-C₃₃-alkylhydroxystearoyl stearates or C₂₀-C₄₀-alkyl erucates. Also suitable are C₃₀-C₅₀-alkyl beeswax, tristearyl citrate, triisostearyl citrate, trilauryl citrate, ethylene glycol dipalmitate, ethylene glycol distearate, ethylene glycol di(12-hydroxystearate), stearyl stearate, palmityl stearate, stearyl behenate, cetearyl behenate, glyceryl mono/dilaurate, -palmitate, -myristate or -stearate, triesters of glycerol with a hydroxystearic acid (Cutina® HR) or glyceryl tristearate, glyceryl tribehenate (e.g. Syncrowax® HRC), glyceryl tripalmitate, or the triglyceride mixtures known by the Syncrowax® HGLC name.

Rheology modifiers present may also be fatty alcohols, fatty acids (unsaponified) and/or glyceryl mono-, di- and/or tri-fatty acid esters and wax esters. Typical examples are cetyl alcohol (Lanette® 16), palmoleyl alcohol, stearyl alcohol (Lanette® 18), cetearyl alcohol (Lanette® O), isostearyl alcohol, oleyl alcohol, elaidyl alcohol, petroselyl alcohol, linolyl alcohol, linolenyl alcohol, elaeostearyl alcohol, arachidyl alcohol, gadoleyl alcohol, behenyl alcohol (Lanette® 22), erucyl alcohol and brassidyl alcohol and technical grade mixtures thereof, which are obtained, for example, in the high pressure hydrogenation of technical grade methyl esters based on fats and oils or aldehydes from Roelen's oxo synthesis and also as monomer fraction in the dimerization of unsaturated fatty alcohols. Particularly preferred fatty alcohols are cetyl alcohol, stearyl alcohol and cetearyl alcohol. Examples of glyceryl esters are glyceryl laurate, such as Monomuls® 90-L-12, glyceryl oleate, such as Monomuls® 90-0 18, glyceryl stearate, such as Cutina® GMS V and Cutina® GMS V/MB, glycol distearate, such as Cutina® AGS, sorbitan stearate, such as Dehymuls® SMS, Cutina® HVG.

Particularly preferred rheology modifiers are those from the group of the polymers anionic, nonionic or cationic, synthetic and/or natural polymers, especially selected from the group formed by polyacrylic acids and alkali metal salts thereof, copolymers of polyacrylic acid and alkali metal salts thereof, copolymers of (meth)acrylic acid and polyether acrylates, where the polyether chain is terminated by a C8-C30-alkyl radical, copolymers of acrylamidomethylpro-panesulfonic acid and alkali metal salts thereof, and the polysaccharides.

From the group of the organic substances, rheology modifiers are more preferably fatty alcohols, glyceryl mono-, di- and/or tri-fatty acid esters, and wax esters.

Especially preferred combinations of the rheology modifiers are mixtures of the hectorites and/or bentonites that have optionally been hydrophobically modified with polymers selected from the group of polyacrylic acids and alkali metal salts thereof, copolymers of polyacrylic acid and alkali metal salts thereof, copolymers of (meth)acrylic acid and polyether acrylates, where the polyether chain is terminated by a C8-C30-alkyl radical, copolymers of acrylamidomethylpropane-sulfonic acid and alkali metal salts thereof and the polysaccharides, and mixtures of the hectorites and/or bentonites that have optionally been hydrophobically modified with organic substances selected from the group of the fatty alcohols, glyceryl mono-, di- and/or tri-fatty acid esters, and wax esters.

### Further Cosmetic Ingredients

The cosmetic ingredient according to the present invention can be selelcted from the group consisting of a stabilizer, a solvent, a solubilizer, a preservative, a neutralizing agent, a buffer and a complexing agent.

### Neutralizing Agents and Buffers

The cosmetic ingredient according to the present invention can be a neutralizing agent or a buffer.

Examples of suitable neutralizing agents are the compatible acids or bases known in the cosmetics industry, which are likewise listed in the Cosmetics Directive. Buffers ensure the pH stability of the cosmetic formulations. Primarily citrate, lactate and phosphate buffers are used.

### Preservatives

The cosmetic ingredient according to the present invention can be a preservative.

Suitable preservatives are, for example, ethanol, isopropanol, phenoxyethanol, the combination of phenoxyethanol with methyldibromoglutaronitrile, formaldehyde solution, parabens, pentanediol or sorbic acid, benzoic acid and salts thereof, Benzyl Alcohol, Benzyl Salicylate, urea condensates, p-hydroxybenzoic esters, dehydroacetic acid, methylthiazolinone or sorbic acid and salts thereof, and also the silver complexes known by the Surfacine® name, and the further substance classes listed in Annex 6, parts A and B, of the Cosmetics Directive. Additionally used are substances which function as preservation aids such as Ethylhexylglycerin and Caprylyl Glycol, and polyols or alcohols such as isopropyl alcohol, propanediol, phenylpropanol, phenethyl alcohol and undecyl alcohol and also the silver complexes known by the Surfacine® name. Additionally suitable as preservatives are the 1,2-alkanediols having 5 to 8 carbon atoms, which are described in WO07/048757.

Advantageous preservatives in the context of the present invention are, for example, formaldehyde releasers (for example DMDM Hydantoin, commercially available, for example, under the Glydant® trade name (Lonza)), iodopropyl butylcarbamate (e.g. Glycacil-L®, Glycacil-S®(Lonza), Dekaben®LMB (Jan Dekker)), parabens (alkyl p-hydroxybenzoate, for example methyl-, ethyl-, propyl- and/or butylparaben), Dehydroacetic Acid (Euxyl® K 702 (Schulke&Mayr), phenoxyethanol, ethanol, benzoic acid. It is also advantageous to use what are called preservation aids, for example octoxyglycerin, glycine, soya etc.

The table below gives an overview of possible preservatives.

| | | | |
|---|---|---|---|
| E200 | Sorbic acid | E227 | Calcium hydrogensulfite |
| E201 | Sodium sorbate | E228 | Potassium hydrogensulfite |
| E202 | Potassium sorbate | E230 | Biphenyl (diphenyl) |
| E203 | Calcium sorbate | E231 | Orthophenylphenol |
| E210 | Benzoic acid | E232 | Sodium |
| | | | orthophenylphenoxide |
| E211 | Sodium benzoate | E233 | Thiabendazole |
| E212 | Potassium benzoate | E235 | Natamycin |
| E213 | Calcium benzoate | E236 | Formic acid |
| E214 | Ethyl p-hydroxybenzoate | E237 | Sodium formate |
| E215 | Ethyl p-hydroxybenzoate | E238 | Calcium formate |
| | Na salt | | |
| E216 | n-Propyl p-hydroxybenzoate | E239 | Hexamethylenetetramine |
| E217 | n-Propyl p-hydroxybenzoate | E249 | Potassium nitrite |
| | Na salt | | |
| E218 | Methyl p-hydroxybenzoate | E250 | Sodium nitrite |
| E219 | Methyl p-hydroxybenzoate | E251 | Sodium nitrate |
| | Na salt | | |
| E220 | Sulfur dioxide | E2li | Potassium nitrate |
| E221 | Sodium sulfite | E280 | Propionic acid |
| E222 | Sodium hydrogensulfite | E281 | Sodium propionate |
| E223 | Sodium disulfite | E282 | Calcium propionate |
| E224 | Potassium disulfite | E283 | Potassium propionate |
| E226 | Calcium sulfite | E290 | Carbon dioxide |

Also advantageous are the preservatives or preservation aids that are commonly used in cosmetics, such as dibromodicyanobutane (2-bromo-2-bromomethylglutarodinitrile), phenoxyethanol, 3-iodo-2-propynylbutyl carbamate, 2-bromo-2-nitropropane-1,3-diol, imidazolidinylu-rea, 5-chloro-2-methyl-4-isothiazolin-3-one, 2-chloroacetamide, benzalkonium chloride, benzyl alcohol, salicylic acid and salicylates.

Particularly preferred preservatives are sodium benzoate, parabens (methyl-, ethyl-, propyl- and/or butylparaben) and/or phenoxyethanol.

### Complexing Agents

The cosmetic ingredient according to the present invention can be complexing agent.

Suitable complexing agents are salts of ethylenediaminetetraacetic acid, of nitrilotriacetic acid, of iminodisuccinic acid, or phosphates.

### Stabilizers

The cosmetic ingredient according to the present invention can be a stabilizer.

Stabilizers can be metal salts of fatty acids, for example magnesium stearate/ricinoleate, aluminum stearate/ricinoleate and/or zinc stearate/ricinoleate, in order to improve emulsion stability, but also compounds such as tris(tetramethylhydroxypiperidinol) citrate as light stabilizers in order to prevent discoloration or changes in odor in the formulations that shall be made.

### Solubilizers

The cosmetic ingredient according to the present invention can be a solubilizer.

Solubilizers can be ethylene oxide adducts having an ethoxylation level of 20 to 60 onto castor oil or hydrogenated castor oil, ethylene oxide adducts and/or propylene oxide adducts having 2-20 ethylene oxide units and 1 to 20 mol of propylene oxide units onto fatty alcohols having 8 to 40 carbon atoms or onto fatty acids having 12 to 40 carbon atoms or onto alkylphenols having 8 to 15 carbon atoms in the alkyl group. Also suitable are ethylene oxide adducts of 1 to 50 mol of ethylene oxide units onto C12-C18 fatty acid mono- and diesters of glycerol or polyglycerol or sorbitan. Preferentially suitable solubilizers are Eumulgin® HRE 40 (INCI: PEG-40 Hydrogenated Castor Oil), Eumulgin® HRE 60 (INCI: PEG-60 Hydrogenated Castor Oil), Eumulgin® L (INCI: PPG-1-PEG-9 Lauryl Glycol Ether), and Eumulgin® SML 20 (INCI: Polysorbate-20).

### Solvents

The cosmetic ingredient according to the present invention can be a solvent.

Suitable solvents are alcohols such as ethanol, propanediol or glycerol.

### Active Cosmetic Ingredients

The cosmetic ingredient according to the present invention can be an active cosmetic ingredient.

Active cosmetic ingredients can be active biogenic ingredients, UV light protection filters, self-tanning agents, insect repellents, antioxidants, film formers, sensory additives, effect pigments, tyrosine inhibitors (depigmenting agents), coolants, perfume oils, dyes and humectants.

### UV Light Protection Filters

The cosmetic ingredient according to the present invention can be a UV light protection filter.

Suitable UV light protection filters organic substances that are liquid at room temperature or crystalline (light protection filters) which are capable of absorbing ultraviolet rays and releasing the energy absorbed again in the form of longer-wave radiation, for example heat. UV filters may be oil-soluble or water-soluble. Examples of typical oil-soluble UV-B filters or broad-spectrum UV-A/B filters include:
3-benzylidenecamphor or 3-benzylidenenorcamphor (Mexoryl SDS 20) and derivatives thereof, e.g. 3-(4-methylbenzylidene)camphor, as described in EP 0693471 B1;
3-(4′-trimethylammonium)benzylidenebornan-2-one methylsulfate (Mexoryl SO);
3,3′-(1,4-phenylenedimethine)bis(7,7-dimethyl-2-oxobicyclo[2.2.1]heptane-1-methanesulfonic acid) and salts (Mexoryl SX);
3-(4′-sulfo)benzylidenebornan-2-one and salts (Mexoryl SL);
polymer of N-{(2 and 4)-[2-oxoborn-3-ylidene)methyl}benzyl]acrylamide (Mexoryl SW);
2-(2H-benzotriazol-2-yl)-4-methyl-6-(2-methyl-3-(1,3,3,3-tetramethyl-1-(trimethylsi-lyloxy)disiloxanyl)propyl)phenol;
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);
esters of salicylic acid, preferably 2-ethylhexyl salicylate, 4-isopropylbenzyl salicylate, ho-momenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, for example 2,4,6-trianilino(p-carbo-2′-ethyl-1′-hexyloxy)-1,3,5-triazine and 2,4,6-tris[p-(2-ethylhexyloxycarbonyl)anilino]-1,3,5-triazine (Uvinul T 150), as described in EP 0818450 A1, or bis(2-ethylhexyl) 4,4′-[(6-[4-((1,1-dimethylethyl)aminocar-bonyl)phenylamino]-1,3,5-triazine-2,4-diyl)diimino]benzoate (Uvasorb® HEB);
2,2-(methylenebis(6-(2H-benzotriazol-2-yl)-4-(1,1,3,3-tetramethylbutyl)phenol) (Tinosorb M);
2,4-bis[4-(2-ethylhexyloxy)-2-hydroxyphenyl]-6-(4-methoxyphenyl)-1,3,5-triazine (Tinosorb S);
propane-1,3-diones, for example 1-(4-tert-butylphenyl)-3-(4′-methoxyphenyl)propane-1,3-dione;
ketotricyclo(5.2.1.0)decane derivatives, as described in EP 0694521 B1;
dimethicodiethyl benzalmalonates (Parsol SLX).

Useful water-soluble UV filters include:
2-phenylbenzimidazole-5-sulfonic acid and the alkali metal, alkaline earth metal, ammonium, alkylammonium, alkanolammonium and glucammonium salts thereof;
2,2-(1,4-phenylene)bis(1H-benzimidazole-4,6-disulfonic acid, monosodium salt) (Neo Heliopan AP);
sulfonic acid derivatives of benzophenones, preferably 2-hydroxy-4-methoxybenzophenone-5-sulfonic acid and salts thereof;
sulfonic acid derivatives of 3-benzylidenecamphor, for example 4-(2-oxo-3-bornylidenemethyl)benzenesulfonic acid and 2-methyl-5-(2-oxo-3-bornylidene)sulfonic acid and salts thereof.

Useful typical UVA filters are especially derivatives of benzoylmethane, for example 1-(4′-tert-butylphenyl)-3-(4′-methoxyphenyl)propane-1,3-dione, 4-tert-butyl-4′-methoxydibenzoylme-thane (Parsol® 1789), 1-phenyl-3-(4′-isopropylphenyl)propane-1,3-dione, and enamine compounds, as described in DE 19712033 A1 (BASF), and also benzoic acid 2-[4-(diethylamino)-2-hydroxybenzoyl]hexyl ester (Uvinul® A plus).

Suitable UV light protection filters are especially the substances approved according to Annex VII of the Commission Directive (in the version: Commission Directive 2005/9/EC of Jan. 28, 2005 amending Council Directive 76/768/EEC, concerning cosmetic products, for the purposes of adapting Annexes VII thereof to technical progress).

In addition to the soluble substances mentioned, insoluble light protection pigments, specifically finely dispersed metal oxides and salts, are also useful for this purpose. Examples of suitable metal oxides are especially zinc oxide and titanium dioxide, and additionally oxides of iron, of zirconium, of silicon, of manganese, of aluminum and of cerium, and mixtures thereof. The salts used may be silicates (talc), barium sulfate or zinc stearate. The oxides and salts are used in the form of the pigments for skincare and skin-protecting emulsions, and also for decorative cosmetics. The particles should have a mean diameter of less than 100 nm, preferably between 5 and 50 nm and especially between 15 and 30 nm. They may have a spherical shape, but it is also possible to use those particles which have an ellipsoidal shape or a shape which deviates in some other way from the spherical configuration. The pigments may also be in surface-treated form, i.e. hydrophilized or hydrophobized. Typical examples are coated titanium dioxides, for example T 805 titanium dioxide (Degussa) or Eusolex® T, Eusolex® T-2000, Eusolex® T-Aqua, Eusolex® AVO, Eusolex® T-ECO, Eusolex® T-OLEO and Eusolex® T-S(Merck). Typical examples are zinc oxides, for example Zinc Oxide neutral, Zinc Oxide NDM (Symrise) or Z-Cote® (BASF) or SUNZnO-AS and SUNZnO-NAS (Sunjun Chemical Co. Ltd.). Suitable hydrophobic coating agents are in particular silicones and specifically trialkoxyoctylsilanes or simethicones. In sunscreen compositions, preference is given to using so-called micropigments or nanopig-ments. Preference is given to using micronized zinc oxide. Further suitable UV light protection filters can be found in the review by P. Finkel in SÖFW-Journal 122, 8/1996, pages 543-548 and Parf.Kosm. Volume 80, no. 3/1999, pages 10 to 16.

As well as the two aforementioned groups of primary photoprotective substances, it is also possible to use secondary photoprotective agents of the antioxidant type which interrupt the photo-chemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthio-uracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to mol/kg), also (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylidene-glucitol, carnosine, butylhydroxytoluene, butylhydroxyanisole, nordihydroguaiacic resin acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids), suitable in accordance with the invention, of these specified active ingredients.

These UV light protection filters are commercially available, for example, under the following trade names:
NeoHeliopan®MBC (INCI: 4-Methylbenzylidene Camphor; manufacturer: Symrise); NeoHeliopan® BB (INCI: Benzophenone-3, manufacturer: Symrise); Parsol®1789 (INCI: Butyl Methox-ydibenzoylmethane, manufacturer: Hoffmann-La Roche (Givaudan); Tinosorb®S (INCI: Bis-Ethylhexyloxyphenol Methoxyphenyl Triazine); Tinosorb®M (INCI: Methylene Bis-Benzotria-zolyl Tetramethylbutylphenol): manufacturer: BASF; Uvasorb®HEB (INCI: Diethylhexyl Butam-ido Triazone, manufacturer: 3V Inc.), Uvinul®T 150 (INCI: Ethylhexyl Triazone, manufacturer: BASF AG); Uvinul®A plus (INCI: Diethylamino Hydroxybenzoyl Hexyl Benzoate, manufacturer: BASF AG; Mexoryl® SO: 3-(4′-trimethylammonium)benzylidenebornan-2-one methylsulfate, INCI: Camphor Benzalkonium Methosulfate; Mexoryl®SX: 3,3′-(1,4-phenylenedi-methine)bis(7,7-dimethyl-2-oxobicyclo-[2.2.1]-heptane-1-methanesulfonic acid), CTFA: INCI Terephthalylidene Dicamphor Sulfonic Acid; Mexory® SL: 3-(4′-sulfo)benzylidenebornan-2-one, INCI Benzylidene Camphor Sulfonic Acid; Mexoryl®SW: polymer of N-{(2 and 4)-[2-oxoborn-3-ylidene)methyl}benzyl]acrylamide, INCI Polyacrylamidomethyl Benzylidene Camphor; Mexoryl®SL: 2-(2H-benzotriazol-2-yl)-4-methyl-6-(2-methyl-3-(1,3,3,3-tetramethyl-1-(trimethylsi-lyloxy)disiloxanyl)propyl)phenol; INCI: DROMETRIZOLE TRISILOXANE; Parsol® SLX: Dime-thicodiethylbenzalmalonate, INCI Polysilicone-15.

Further examples are pigmentary light filters, for example inorganic pigmentary light filters, such as titanium dioxide and zinc oxide and/or organic pigmentary light filters such as meth-ylenebisbenzotriazolyltetramethylbutylphenol (Tinosorb M).

### Self-Tanning Agents

The cosmetic ingredient according to the present invention can be a self-tanning agent.

Self-tanning agents are understood to mean substances which cause browning of the skin. Examples include dihydroxyacetone, erythrulose and alpha, beta-unsaturated aldehydes, which react with the amino acids in the skin in the manner of a Maillard reaction to give colored compounds. Useful further active ingredients for self-tanning agents include natural or synthetic ke-tols or aldols. Examples of suitable active ingredients include dihydroxyacetone, erythrulose, glycerolaldehyde, alloxane, hydroxymethylglyoxal, gamma-dialdehyde, 6-aldo-D-fructose, nin-hydrin and meso-tartaraldehyde. Suitable self-tanning agents are especially dihydroxyacetone and/or erythrulose.

### Active Biogenic Ingredients

The cosmetic ingredient according to the present invention can be an active biogenic ingredient.

Active biogenic ingredients can be active ingredients having a natural basis, preferably those that improve skin properties, for example tocopherol, tocopherol acetate, tocopherol palmitate, ascorbic acid, (deoxy)ribonucleic acid and the fragmentation products thereof, β-glucans, retinol, bisabolol, allantoin, phytantriol, panthenol, AHA acids, amino acids, ceramides, pseudocer-amides, essential oils, plant extracts, for example aloe vera, prunus extract, bambara nut extract and vitamin complexes.

The following active biogenic ingredients are suitable as humectants for the skin:
AMC® Advanced Moisture Complex NP (INCI: Glycerin (and) Water (and) Sodium PCA (and) Urea (and) Trehalose (and) Hexylene glycol (and) Polyquaternium-51 (and) Triacetin (and) Caprylyl Glycol (and) Sodium Hyaluronate); Hyalurosmooth® LS8998 (INCI: Water (and) Cassia Angustifolia Seed Polysaccharide); Hyalurosmooth® LS 8997(INCI: Cassia Angustifolia Seed Polysaccharide); Irwinol® (INCI: Octyldodecanol (and) Irvingia Gabonensis Kernel Butter (and) Hydrogenated Coco-Glycerides); Lipofructyl® Argan (INCI: Argania Spinosa Kernel Oil); Melhydran® (INCI: Water (and) Butylene Glycol (and) Honey Extract (and) Glycerin (and) Urea (and) Sodium Lactate (and) Arginine HCl (and) Lysine HCl (and) Ornithine HCl); Oligolin® (INCI: Hydrolyzed Linseed Extract); PatcH₂O® (INCI: Water (and) Glycerin (and) Trehalose (and) Urea (and) Serine (and) Pentylene Glycol (and) Glyceryl Polyacrylate (and), Algin (and), Caprylyl Glycol (and) Sodium Hyaluronate (and), Pullulan (and) Disodium Phosphate (and) Potassium Phosphate); Relipidium® (INCI: Hydrolyzed Yeast Protein (and) Butylene Glycol (and) Pentylene Glycol); Sphingoceryl® VEG (INCI: Octyldodecanol (and) Hydrogenated Coco-Glycerides (and) Helianthus Annuus (Sunflower) Seed Extract).

The following active biogenic ingredients are suitable as anti-aging agents:
AH-Care® (INCI: Water (and) LacticAcid (and) Arginine); Argassential® (INCI: Argania Spinosa Fruit Extract (and) Dicaprylyl Ether (and) Sorbitol (and) Polyglyceryl-2 Dipolyhydroxystearate (and) Lauryl Glucoside (and) Glycerin (and) Water); Argatensyl® (INCI: Argania Spinosa Kernel Extract (and) Sodium Cocoyl Glutamate); Collalift® 18 (INCI: Glycerin (and) Water (and) Khaya Senegalensis Bark Extract (and) Maltodextrin); Deliner® (INCI: Water (and) Butylene Glycol (and) Pentylene Glycol (and) Zea Mays (Corn) Kernel Extract (and) Xanthan Gum); Dermagen-ist® (INCI: Maltodextrin (and) Origanum Majorana Leaf Extract); Dermican® LS 9837 (INCI: Glycerin (and) Water (and) Acetyl Tetrapeptide-9); Dermican® LS 9838 (INCI: Mannitol (and) Acetyl Tetrapeptide-9); Elestan® LS 9913 (INCI: Glycerin (and) Manilkara Multinervis Leaf Extract (and) Water); Elestan® LS 9879 (INCI: Manilkara Multinervis Leaf Extract (and) Maltodextrin); Eperuline® (INCI: Maltodextrin (and) Eperua Falcata Bark Extract); Epigenist® (INCI: Maltodextrin (and) Voandzeia Subterranea Seed Extract); Hyalufix® (INCI: Water (and) Butylene Glycol (and) Alpinia Galanga Extract (and) Pentylene Glycol (and) Xanthan Gum (and) Caprylic/Capric Triglyceride), Hyaluronic Filling Spheres® (INCI: Ethylhexyl Palmitate (and) Tri-hydroxystearin (and) Sodium Hyaluronate); Linefactor® C (INCI: Water (and) Butylene Glycol (and) Pentylene Glycol (and) Hibiscus Abelmoschus Extract (and) Xanthan Gum); Lox-Age® (INCI: Water (and) Cichorium Intybus (Chicory) Leaf Extract (and) Hexylene Glycol (and) Caprylyl Glycol (and) Xanthan Gum); Lys'lastine® V (INCI: Water (and) Butylene Glycol (and) Peucedanum Graveolens (Dill) Extract (and) Pentylene Glycol (and) Xanthan Gum); Myoxinol® (INCI: Hydrolyzed Hibiscus Esculentus Extract (and) Dextrin); Neurobiox® (INCI: Water (and) Butylene Glycol (and) Pentylene Glycol (and) Achillea Millefolium Extract (and) Xanthan Gum); Oligolin® (INCI: Hydrolyzed Linseed Extract); Perlaura® (INCI: Water (and) Polygonum Bistorta Root Extract (and) Hexylene Glycol (and) Caprylyl Glycol (and) Xanthan Gum); Phytokine® (INCI: Hydrolyzed Soy Protein (and) Butylene Glycol (and) Pentylene Glycol (and) 1,2-Hex-anediol (and) Caprylyl Glycol); Proteasyl® LS 9818 (INCI: Water (and) Glycerin (and) Pisum Sativum (Pea) Extract); Proteasyl® LS 8951 (INCI: Pisum Sativum (Pea) Extract (and) Cy-clodextrin); Shadownyl® (INCI: Water (and) Fucus Vesiculosus Extract (and) Hexylene Glycol (and) Caprylyl Glycol (and) Xanthan Gum); Slim-Excess® (INCI: Water (and) Butylene Glycol (and) Pentylene Glycol (and) Sodium Chloride (and) Hydrolyzed Rhodophycea Extract (and) Xanthan Gum); Speci'Men® (INCI: Glycerin (and) Water (and) Hydrolyzed Adansonia Digitata Extract); Sqisandryl® (INCI: Schizandra Chinensis Fruit Extract); Syniorage® (INCI: Mannitol (and) Acetyl Tetrapeptide-11); Ultra Filling Sphreres® (INCI: Ethylhexyl Palmitate (and) Trihy-droxystearin (and) Sodium Hyaluronate (and) Glucomannan); Vegeseryl® HGP (INCI: Water (and) Glycine Soja (Soybean) Protein (and) Sodium Cocoyl Glutamate (and) Ethylhexylglycerin); Vit-A-Like® LS 9793 (INCI: Water (and) Vigna Aconitifolia Seed Extract (and) Sodium Cocoyl Glutamate); Vit-A-Like® LS 9898 (INCI: Vigna Aconitifolia Seed Extract (and) Maltodextrin); X-pressin® R (INCI: Water (and) Papain (and) Carbomer (and) Caprylyl Glycol (and) Algin).

Suitable active biogenic ingredients for improving the skin (skin perfection) are:
AH-Care® (INCI: Water (and) Lactic Acid (and) Arginine); Beta-Hydroxyde® ACSD (INCI: Salicylic Acid (and) Acacia Senegal Gum); Betapur® (INCI: Water (and) Butylene Glycol (and) Peumus Boldus Leaf Extract (and) Pentylene Glycol (and) Xanthan Gum); Biophytex® (INCI: Water (and) Butylene Glycol (and) Panthenol (and) Escin (and) Glycerin (and) Ruscus Aculeatus Root Extract (and) Ammonium Glycyrrhizate (and) Centella Asiatica Leaf Extract (and) Hydrolyzed Yeast Protein (and) Calendula Officinalis Flower Extract); Lox-Age® (INCI: Water (and) Cichorium Intybus (Chicory) Leaf Extract (and) Hexylene Glycol (and) Caprylyl Glycol (and) Xanthan Gum); Mat-XS® Clinical (INCI: Water (and) Butylene Glycol (and) Pentylene Glycol (and) Sarcosine (and) Xanthan Gum); Neurobiox® (INCI: Water (and) Butylene Glycol (and) Pentylene Glycol (and) Achillea Millefolium Extract (and) Xanthan Gum; X-pressin® C (INCI: Water (and) Papain (and) Carbomer (and) Caprylyl Glycol (and) Algin).

### Humectants

The cosmetic ingredient according to the present invention can be a humectant. Humectants can bind water and hence prevent evaporation. Suitable humectants are polyols having 2 to 10 carbon atoms and 2 to 6 hydroxyl groups, for example glycerol, sorbitol, propylene glycol, butylene glycol, polyethylene glycols or xylitol.

### Insect Repellents

The cosmetic ingredient according to the present invention can be an insect repellent.

Examples of useful insect repellents include N,N-diethyl-m-toluamide, 1,2-pentanediol or ethyl 3-(N-n-butyl-N-acetylamino)propionate, which is sold under the Insect Repellent® 3535 name by Merck KGaA, and butyl acetylaminopropionates.

### Tyrosine Inhibitors

The cosmetic ingredient according to the present invention can be a tyrosine inhibitor.

Examples of useful tyrosine inhibitors which prevent the formation of melanin and find use in depigmenting agents include arbutin, ferulic acid, kojic acid, coumaric acid and ascorbic acid (vitamin C).

### Perfume Oils

The cosmetic ingredient according to the present invention can be a perfume oil

Perfume oils include mixtures of natural and synthetic odorants. Natural odorants are extracts from flowers (lily, lavender, rose, jasmine, neroli, ylang ylang), stems and leaves (geranium, patchouli, petitgrain), fruits (aniseed, coriander, caraway, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, for example civet and castoreum. Typical synthetic odorant compounds are products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include primarily the terpenes and balsams. However, preference is given to using mixtures of different odorants which together produce a pleasant scent note. Essential oils of relatively low volatility, which are mostly used as aroma components, are also suitable as perfume oils, e.g. sage oil, chamomile oil, clove oil, melissa oil, mint oil, cinnamon leaf oil, linden blossom oil, juniper berry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillate, irotyl and floramate alone or in mixtures. Examples of suitable aromas include peppermint oil, spearmint oil, aniseed oil, star anise oil, caraway oil, eucalyptus oil, fennel oil, lemon oil, wintergreen oil, clove oil, menthol and the like.

### Film Formers

The cosmetic ingredient according to the present invention can be a film former.

Suitable film formers are, for example, chitosan, microcrystalline chitosan, quaternized chitosan, polyvinylpyrrolidone, vinylpyrrolidone-vinyl acetate copolymers, quaternary cellulose derivatives, collagen, hyaluronic acid and salts thereof and similar compounds.

### Dyes

The cosmetic ingredient according to the present invention can be a dye.

Dyes can be the substances approved and suitable for cosmetic purposes, as listed, for example, in the publication “Kosmetische Färbemittel” [Cosmetic Colorants] from the Farbstoffkom-mission der Deutschen Forschungsgemeinschaft [Dyes Commission of the German Research Society], Verlag Chemie, Weinheim, 1984, pp. 81-106. Examples are cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). As a luminescent dye, it is also possible for luminol to be present. These dyes are usually used in concentrations of from 0.001% to 0.1% by weight, based on the total mixture.

### Effect Pigments

The cosmetic ingredient according to the present invention can be an effect pigment.

The preferred particle size of the pigments is 0.01 to 200 μm, especially 0.02 to 150 μm, more preferably 0.05 to 100 μm.

The pigments are colorants that are virtually insoluble in the application medium and may be inorganic or organic. Inorganic-organic mixed pigments are also possible. Preference is given to inorganic pigments.

The advantage of the inorganic pigments is their excellent photostability, weather stability and thermal stability. The inorganic pigments may be of natural origin, for example prepared from chalk, ochre, umber, green earth, burnt siena or graphite. The pigments may be white pigments, such as, for example, titanium dioxide or zinc oxide, black pigments, such as, for example, iron oxide black, colored pigments, such as, for example, ultramarine or iron oxide red, gloss pigments, metal effect pigments, pearlescent pigments and fluorescent or phosphorescent pigments, where preferably at least one pigment is a colored, non-white pigment.

Metal oxides, hydroxides and oxide hydrates, mixed-phase pigments, sulfur-containing silicates, metal sulfides, complex metal cyanides, metal sulfates, chromates and molybdates, and the metals themselves (bronze pigments) are suitable. Of particular suitability are titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), iron blue (ferric ferrocyanide, CI 77510), carmine (cochineal).

Particular preference is given to pearlescent pigments and colored pigments based on mica or based on borosilicate, which are coated with a metal oxide or a metal oxychloride, such as titanium dioxide or bismuth oxychloride, and if appropriate further color-imparting substances, such as iron oxides, iron blue, ultramarine, carmine etc., and where the color can be determined by varying the layer thickness. Such pigments are sold, for example, under the Rona®, Colorona®, Dichrona® and Timiron® trade names by Merck or the Reflecks® Cloisonn6®, Timica®, Chione®, Pearl-Glo® or Bi-Lite® trade names by BASF.

Organic pigments are, for example, the natural pigments sepia, gamboge, bone charcoal, Cassel brown, indigo, chlorophyll and other plant pigments.

Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

Especially suitable are, for instance, silica, silicates, aluminates, aluminas, mica, salts, especially organic metal salts, metal oxides, e.g. titanium dioxide, optionally in combination with fluorescent or phosphorescent pigments.

### Sensory Additives

The cosmetic ingredient according to the present invention can be a sensory additive. Examples of suitable sensory additives include Aluminum Starch Octenylsuccinate, Tapoca, Dimethicone and Dimethicone Crosspolymer or Polymethyl silsequioxane.

### Coolants

The cosmetic ingredient according to the present invention can be a coorant. Examples of suitable coolants include ethanol, menthol and/or camphor.

The chemical production network may include one or more one or more chemical and/or mechanical process(es). The chemical production network may produce one or more output material(s) through chemical and/or mechanical processing. The chemical production network may include multiple types of production processes for producing one or more output material(s) from one or more input material(s). The chemical production network may produce one or more output material(s) from input material(s) provided to the chemical production network. The chemical production network may include a complex production network producing multiple chemical products via multiple production process(es). The chemical production network may include connected, interconnected and/or non-connected production process(es). The chemical production network may include a composite or Verbund network.

The chemical production network may include identity preserving or segregated production process(es). Identity preserving or segregated in this context may refer to environmental attribute(s) of input material(s) being preserved or segregated in the production process(es). Examples are non-fossil, e.g. renewable or recycled, input materials used to produce the one or more cosmetic ingredient(s) without fossil content. Further examples are fossil input material(s) used to produce the one or more cosmetic ingredient(s) with fossil content. Chemical production networks may include non-identity preserving or non-segregated production process(es). Non-identity preserving or non-segregated in this context may refer to non-fossil input material(s) being mixed with fossil input material(s) to produce the cosmetic ingredient(s). For example, fossil and renewable input materials may be mixed to produce the cosmetic ingredient(s) with fossil and renewable content.

The chemical production network may include one or more production process(es) with multiple production steps. The production steps included in the chemical network may be defined by the physical system boundary of the chemical production network. The system boundary may be defined by location and/or control over production processes or steps. The system boundary may be defined by a site of the chemical production network. The system boundary may be defined by production process(es) or step(s) controlled by one entity or multiple entities jointly. The system boundary may be defined by the value chain with staggered production process(es) or step(s) to the chemical end product, which may be controlled by multiple entities jointly or separately. The chemical production network may include a waste collection, a sorting step, a recycling step such as pyrolysis, a cracking step such as steam cracking, a separation step to separate intermediates of one process step and further processing steps to convert such intermediates to output material(s), in particular the produced cosmetic ingredient(s) leaving the system boundary of the chemical production network. The input material(s) may enter the physical system boundary of the chemical production network. The entry point(s) of the chemical production network may be marked by the entry of input material(s) to the chemical production network or the system boundary of the chemical network. The output material(s), in particular the produced cosmetic ingredient(s), may leave the physical system boundary of the chemical production network. The exit point(s) of the chemical production network may be marked by the exit of output material(s), in particular the produced cosmetic ingredient(s), from the chemical production network or the system boundary of the chemical network.

The chemical production network may include one or more production chain(s) for the production of cosmetic ingredients. The production chain(s) for the production of cosmetic ingredients may be interconnected. The production chain(s) for the production of cosmetic ingredients may be interconnected with production chain(s) for the production of other output material(s). The production chain(s) for the production of cosmetic ingredients may include production chain(s) for the production of intermediates used to produce cosmetic ingredients. The production chain(s) for the production of cosmetic ingredients may use input material(s) provided by chemical network(s) for the production of intermediates usable to produce cosmetic ingredients.

One or more input material(s) may be provided to the chemical production network for producing one or more output material(s), in particular cosmetic ingredient(s). The output material(s), in particular cosmetic ingredient(s), may be produced from one or more input material(s) through one or more chemical process(s) of the chemical production network. The input material may comprise any input material entering the chemical production network at any entry point. The input material may include organic or inorganic input material(s). The input material may be a pre-cursor product, an intermediate material, a feedstock or a raw material used to produce one or more output material(s), in particular cosmetic ingredient(s). The input material may be fed to the chemical production network to produce one or more output material(s), in particular cosmetic ingredient(s). The input material may be fed to chemical production network including one or more production process(es) with multiple process steps. The input material may be fed to the chemical production network at the start of the production process or at any intermediate stage of the production process. The input materials entering the chemical production network may be used to produce one or more output material(s), in particular cosmetic ingredient(s).

The input material may be associated with an input material identifier. The input material identifier may comprise any identifier uniquely associated with the input material. The input material identifier may be associated with the physical entity of the input material. The input material identifier may be associated with a single batch of input material. The input material identifier may be associated with a group of input materials. The identifier may be associated with multiple physical entities of the input material. The input material identifier may be associated with continuous or semi-continuous stream of input material. The input material identifier may be associated with a stream of the input material e.g. over a certain time period or from a certain supplier. The input material identifier may be linked or connected to one or more environmental attribute(s).

Environmental attribute may refer to a property related to the environmental impact. Such property may be the property of input material(s), chemical process(es), chemical production network(s) and/or cosmetic ingredient(s). The environmental attribute may indicate an environmental performance of input material(s), chemical process(es), chemical production network(s) and/or cosmetic ingredient(s). The environmental attribute may be derived from properties of input material(s), chemical process(es), chemical production network(s) and/or cosmetic ingredient(s). The environmental attribute may be associated with the environmental impact of input material(s), chemical process(es), chemical production network(s) and/or cosmetic ingredient(s) at any stage of the lifecycle of the input material(s) and/or cosmetic ingredient(s). The stages may include providing raw material, providing feedstock, producing chemical products, such as intermediate products or end products, producing discrete products by using the chemical products, using chemical products or discrete products, treating end-of-life products, recycling end-of-life products, disposing end-of-life products, reusing components from end-of-life products or any subset of stages. The environmental attribute may be specified or may be derived from any activity of one or more entities participating at any stage of the lifecycle of one or more material(s) or product(s).

The environmental attribute may include one or more characteristic(s) that are attributable to environmental impact of input material(s), chemical process(es), chemical production network(s) and/or cosmetic ingredient(s). The environmental attribute may include environmental, technical, recyclability or circularity characteristics(s) associated with the environmental impact of input material(s), chemical process(es), chemical production network(s) and/or cosmetic ingredient(s).

The environmental attribute may include one or more characteristic(s) that are attributable to the environmental impact of input material(s), chemical process(es), chemical production network(s) and/or cosmetic ingredient(s). The environmental attribute may include environmental, technical, recyclability or circularity characteristics(s) associated with the environmental impact of input material(s), chemical process(es), chemical production network(s) and/or cosmetic ingredient(s). The one or more environmental attribute(s) may be attributable to the environmental impact of the cosmetic ingredient. The one or more environmental attribute(s) may relate to environmental, technical, recyclability, circularity and/or complementary risk characteristic(s) of the cosmetic ingredient.

Environmental characteristic(s) may specify or quantify ecological criteria associated with environmental impact. Environmental characteristic(s) may be or may be derived from measurements taken during the lifecycle. Environmental characteristics may be determined at any stage of the lifecycle and may characterize the environmental impact for such stage or up to such stage. Environmental characteristic(s) may for example include carbon footprint, greenhouse gas emissions, resource usage, air emissions, ozone depletion potential, water pollution, noise pollution, energy consumption, waste reduction, or eutrophication potential. Environmental characteristic(s) may for example include product characteristics related to the production of the product like bio based, vegetable based, animal based, halogen-free, fluorine-free, vegan, halal, kosher, palm oil-free, natural, tox-fee, volatile organic compounds-free or any combinations thereof.

Technical characteristic(s) may specify or quantify performance at least indirectly associated with the environmental impact. Technical characteristic(s) may be or may be derived from measurements taken during the lifecycle. Technical characteristics may be determined at any stage of the lifecycle and may characterize the performance for such stage or up to such stage. Technical characteristic(s) may for example include chemical composition data, raw material composition such as bio-based or recycled input material content specifying e.g. x % non fossil and y % fossil content, bill of materials, product specification data such as product purity, product form (as indication to their impact on dust formation/release), safety data, product extractability, migration data, toxicological data or ecotoxicological data, product component data, safety data, application property data, application instructions, quality data or any combinations thereof.

Circularity characteristic(s) may specify or quantify the life cycle characteristics associated with circular uses. Circularity characteristic(s) may be or may be derived from measurements taken during the lifecycle. Circularity characteristic(s) may be or may be derived from circular data recorded in one or more prior lifecycle(s) including reuse. Circularity characteristic(s) may be determined at any stage of the lifecycle and may characterize the reuse or recycling performance for such stage or up to such stage. Circularity characteristic(s) may for example include recycling data, reuse rate, recycling rate, recycling loops, reuse performance, reused quality or any combinations thereof.

Recyclability characteristic(s) may specify or quantify life cycle characteristics associated with recycling uses. Recyclability characteristic(s) may include the composition of the material including specifically tailored constituents making the material suitable for recycling. Recyclability characteristic(s) may be or may be derived from measurements taken during the lifecycle. Recyclability characteristic(s) may be or may be derived from recycling data recorded in one or more prior lifecycle(s). Recyclability characteristics may be determined at any stage of the lifecycle and may characterize the recycling performance for such stage or up to such stage. Recyclability characteristic(s) may for example include recycling data, number of reuses, recyclate composition, recyclate quality, waste stream composition, waste stream quality or any combinations thereof.

In one embodiment the cosmetic ingredients passport or digital asset associated with the cosmetic ingredient may include mass balanced environmental attributes related to the input material. Mass balanced environmental attributes may include environmental attributes of the input material(s) used to produce the cosmetic ingredient, which are tracked and by mass attributable to the cosmetic ingredient. The environmental impact of input material(s) may be determined based on input material(s) used in the chemical process(s) to produce the cosmetic ingredient. For example, bio-based, renewable and/or recycled content of input material(s) used to produce the cosmetic ingredient may be tracked. Further for example, castor oil or palm oil content of input material(s) used to produce the cosmetic ingredient may be tracked. Further for example, properties related to the environmental impact of the input material include RSPO palm oil, palm oil free or castor oil may be tracked. Further for example, properties of the chemical process(es) used to produce the cosmetic ingredient may be tracked. Examples of tracked process properties related to the environmental impact include water consumption, CO₂ emissions and/or Greenhouse Gas (GHG) emissions, amount of waste generation, mixed material generation, design for recycling, energy consumption, processing properties such as less waste or less loss of properties. The properties may be tracked based on a certificate from a certifying agency. The properties may be tracked based on inherent physical properties derived from measurements.

In one embodiment the produced cosmetic ingredient is connected to the decentral identifier physically identifying the produced cosmetic ingredient. The production operating apparatus may be configured to provide the decentral identifier associated with a physical entity of the produced cosmetic ingredient. The production operating apparatus may be configured to link the decentral identifier to a physical identifier of the produced cosmetic ingredient. The production operating apparatus may be configured to assign the decentral identifier to the physical identifier connected to the produced cosmetic ingredient. The production operating apparatus may be configured to assign the decentral identifier to the physical identifier physically connected to the produced cosmetic ingredient.

In one embodiment the decentral identifier relates to data associated with at least one product produced from the cosmetic ingredient, wherein the one or more environmental attribute(s) associated with the at least one product is derived from one or more environmental attribute(s) associated with the cosmetic ingredient. The one or more environmental attribute(s) associated with the cosmetic ingredient may be associated with the one or more input material(s) and/or the chemical process(s) used to produce the cosmetic ingredient. The decentral identifier may relate to any identifier uniquely associated with the cosmetic ingredient. The decentral identifier may be associated with the physical entity of the cosmetic ingredient. The decentral identifier may refer to a single batch of cosmetic ingredient. The decentral identifier may be associated with a group of cosmetic ingredients. The identifier may refer to multiple physical entities of the cosmetic ingredient. The decentral identifier may be associated with continuous or semi-continuous stream of cosmetic ingredient. The identifier may refer to a stream of the cosmetic ingredient e.g. over a certain time period or from a certain supplier.

In one embodiment the one or more environmental attribute(s) associated with the cosmetic ingredient(s) are provided from at least one balancing account configured to store environmental attribute(s) associated with input material(s). The balancing account may relate to storage structure associated with metadata, such as an environmental attribute type. For instance, the balancing account may be associated with metadata indicating the environmental attribute type to be recycled-content or bio-based. An inbound allocator may be configured to allocate the one or more environmental attribute(s) associated with input material(s) to at least one balancing account e.g. on entry of the input material to the chemical production network. The balancing account may be associated with the respective environmental attribute type. An outbound assigner may be configured to assign at least one environmental attribute from the at least one balancing account associated with the respective environmental attribute to the decentral identifier. One or more environmental attribute(s) may be assigned to the at least one decentral identifier. Assignment may include de-allocation of the one or more environmental attributes from the balancing account associated with the respective environmental attribute type. By using the balancing accounts environmental attributes of input materials can be reliably tracked and assigned to cosmetic ingredients.

In one embodiment the one or more environmental attribute(s) associated with the cosmetic ingredient(s) are provided from at least one balancing account configured to store environmental attribute(s) associated with input material(s). An inbound allocator may be configured to allocate the one or more environmental attribute(s) to at least one balancing account associated with the respective environmental attribute, e.g. on input material entering the chemical production network. The balancing account may be associated with the respective environmental attribute type. The one or more environmental attribute(s) associated with the input materials may be allocated to the balancing account associated with the respective environmental attribute type. An outbound assigner may be configured to assign or link at least one environmental attribute from the at least one balancing account associated with the respective environmental attribute type to the decentral identifier. This may include de-allocation of the one or more environmental attribute(es) from the balancing account associated with the respective environmental attribute type. By using the balancing accounts environmental attributes can be tacked through the chemical production network. This way the environmental attributes may be detached from the material flow. Attribution of environmental attribute(s) may be conducted on a mass balance basis for the chemical production network. In such approach the total mass of input materials and cosmetic ingredients as well as the attribution of respective environmental attribute(s) associated with input materials and cosmetic ingredients are balanced.

In one embodiment the one or more environmental attribute(s) are associated with at least one property related to the environmental impact of the one or more input material(s) and/or the chemical process(s). The one or more environmental attribute(s) may specify environmental properties of the input material(s) used to produce the cosmetic ingredient and/or the one or more environmental attribute(s) may specify environmental properties of the chemical process(es) used to produce the cosmetic ingredient. The one or more environmental attribute(s) may be generated from environmental properties of the input material(s) used to produce the cosmetic ingredient, process data associated with the chemical processing of the input material(s) and/or energy data associated with the energy consumption of the chemical processing. The one or more environmental attribute(s) may include a recycled content associated with the input material(s) and allocated or allocatable to the cosmetic ingredient(s), a renewable content associated with the input material(s) and allocated or allocatable to the cosmetic ingredient(s), and/or a product carbon footprint associated with the cosmetic ingredient(s).

In one embodiment the production operating apparatus is configured to gather environmental attributes associated with the produced cosmetic ingredient before, during and/or after production of the cosmetic ingredient by the chemical production network. The environmental attributes associated with the produced cosmetic ingredient may relate to input material(s). The environmental attributes associated with input materials may be provided before, during and/or after production of the cosmetic ingredient by the chemical production network. The environmental attributes associated with input materials may be allocated to at least one balancing account before, during and/or after production of the cosmetic ingredient by the chemical production network. The environmental attributes associated with the produced cosmetic ingredient may relate to environmental properties generated from process data associated with the chemical processing of the input material(s) and/or energy data associated with the energy consumption of the chemical processing. The environmental attributes associated with the produced cosmetic ingredient may be generated before, during and/or after production of the cosmetic ingredient by the chemical production network. The process data associated with the chemical processing of the input material(s) and/or energy data associated with the energy consumption of the chemical processing may be gathered prior, during and/or after production of the cosmetic ingredient.

In one embodiment the cosmetic ingredient passport or the digital asset may include the decentral identifier associated with the cosmetic ingredient and the one or more environmental attribute(s) linked to the decentral identifier. The one or more environmental attribute(s) may be linked to the decentral identifier included in the cosmetic ingredient passport or the digital asset. The one or more environmental attribute(s) may be stored in a data base associated with or of the cosmetic ingredient producer for access by any consumer of the cosmetic ingredient. The one or more environmental attribute(s) may be stored in a data base associated with or of the cosmetic ingredient producer for transfer to a consume of the cosmetic ingredient e.g. when accessed or on providing the cosmetic ingredient. The decentral identifier may comprise any unique identifier uniquely associated with the cosmetic ingredient producer and cosmetic ingredient data such as the environmental attributes. The decentral identifier may include a Universally Unique IDentifier (UUID) ora Digital IDentifier(DID). The decentral identifier may be issued by a central or decentral identity issuer. The decentral identifier may be linked to authentication and/or authorization information. Via the decentral identifier and its unique association with the cosmetic ingredient producer and cosmetic ingredient data, such as the environmental attributes, access to the cosmetic ingredient data may be controlled by the cosmetic ingredient producer. This contrasts with central authority schemes, where identifiers are provided by such central authority and access to data is controlled by such central authority. Decentral in this context refers to the usage of the identifier in implementation as controlled by the data owner, such as the cosmetic ingredient producer.

The decentral identifier may be uniquely associated with the cosmetic ingredient or the physical entity of the cosmetic ingredient, e.g. as packaged for transportation to the consumer of the cosmetic ingredient. The decentral identifier may be uniquely to the one or more environmental attribute(s). The cosmetic ingredient passport or the digital asset may include one or more digital representation(s) pointing to cosmetic ingredient data including the environmental attribute(s) or parts thereof including the environmental attribute(s). The digital representation may comprise at least one interface to a data providing service. It may further include at least one interface to a data consuming service. It may include an endpoint for data exchange or sharing (resource endpoint) or an endpoint for service interaction (service endpoint), that is uniquely identified via a communication protocol. The digital representation(s) pointing to cosmetic ingredient data or parts thereof may be uniquely associated with the decentral identifier.

The cosmetic ingredient passport or the digital asset may comprise or be connected to a digital representation of cosmetic ingredient data, such as environmental attribute(s). The digital representation may include a representation for accessing the cosmetic ingredient data, such as environmental attribute(s) or part thereof. The digital representation may include a representation of cosmetic ingredient data, such as environmental attribute(s). The cosmetic ingredient passport or the digital asset may include or be connected to data related to the cosmetic ingredient data, such as environmental attribute(s), the authentication information and the decentral identifier. The data related to the cosmetic ingredient data, such as environmental attribute(s), may include the digital representation of the cosmetic ingredient data, such as environmental attribute(s).

### BRIEF DESCRIPTION OF THE DRAWINGS

In the following, the present disclosure is further described with reference to the enclosed figures:

FIG. 1 illustrates schematically an example of a chemical production network producing one or more output material(s) from one or more input material(s) in connection with a production operating system including an attribute management system.

FIG. 2 illustrates schematically an example of attributing environmental attributes of input materials to output materials of the chemical production network.

FIG. 3 illustrates schematically an example of attributing environmental attributes of input materials and chemical processes to an output material of the chemical production network.

FIG. 4 illustrates schematically another example of a method or apparatus for providing environmental attributes associated with output materials to a material user as data consumer via a decentral network.

FIG. 5 illustrates schematically an example of a method or apparatus for providing environmental attributes of output materials across value chains via the decentral network.

FIG. 6 illustrates schematically an example of a chemical production network for producing cosmetic ingredients associated with the digital asset.

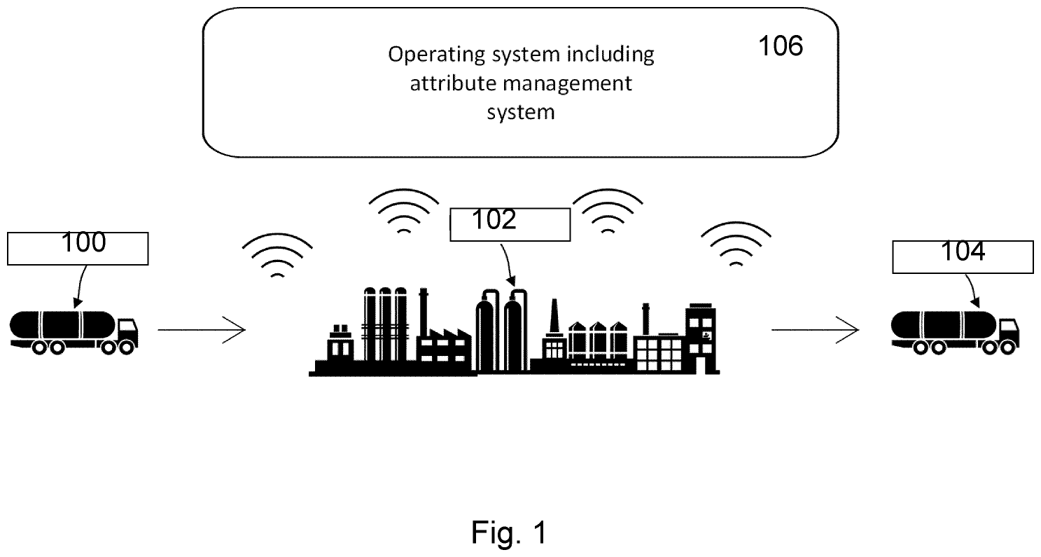

FIG. 1 illustrates an example of a chemical production network 102 producing one or more output material, in particular cosmetic ingredient(s) 104 from one or more input material(s) 100 in connection with a production operating system 106 including an attribute management system.

For producing one or more output material(s), in particular cosmetic ingredient(s)104 different input materials 100 may be provided as physical inputs to the chemical production network 102. The physical input material(s) 100 and output material(s), in particular cosmetic ingredient(s), 104 may be associated with one or more properties related to environmental impact. The properties related to environmental impact may be digitalized in the form of environmental attributes such as recycled or bio-based content of the input materials. The production operating system 106 may be configured to ingest such environmental attributes and to track the environmental attributes across the chemical production network 102 from input materials 100 to output material(s), in particular cosmetic ingredient(s), 104.

The chemical production network 102 may include multiple interlinked processing steps. The chemical production network 102 may be an integrated chemical production network 102 with interrelated production chains. The chemical production network 102 may include multiple different production chains that have at least one intermediate product in common. The chemical production network 102 may include multiple stages of the chemical value chain. The chemical production network 102 may include the producing, refining, processing and/or purification of gas or crude oil. The chemical production network 102 may include a stream cracker, or a syngas plant connected to multiple production chains that output products 104 from the effluent of such plants. The chemical production network 102 may include multiple production chains that output from one or more input material(s) 100 one or more output material(s), in particular cosmetic ingredient(s), 104. The chemical production network 102 may include multiple tiers of a chemical value chain. The chemical production network 102 may include a physically interconnected arrangement of production sites. The production sites may be at the same location or at different locations. In the latter case the production sites may be interconnected by means of dedicated transportation systems such as pipelines, supply chain vehicles, like trucks, supply chain ships or other cargo transportation means.

The chemical production network 102 may chemically convert input materials 100 to one or more output material(s), in particular cosmetic ingredient(s), 104. The chemical production network 102 may convert input materials 100 by way of chemical conversion to one or more output material(s), in particular cosmetic ingredient(s), 104.

The input materials 100 may be fed to the chemical production network 102 at any entry point. The input materials 100 may be fed to the chemical production network 102 at the start of the chemical production network 102. Input materials 100 may for example make up the feedstock of a steam cracker. The input material 100 may include non-fossil input material, such as bio-based or recycled material, and/or fossil input material for the manufacture of chemical intermediates and chemical output material(s), in particular cosmetic ingredient(s), 104.

The chemical production network 102 may include multiple production steps. The production steps included in the chemical production network 102 may be defined by the system boundary of the chemical production network 102. The system boundary may be defined by location or control over production processes. The system boundary may be defined by the site of the chemical production network 102. The system boundary may be defined by production processes controlled by one entity or multiple entities jointly. The system boundary may be defined by value chain with staggered production processes to an end product, which may be controlled by multiple entities separately. The chemical production network 102 may include a waste collection and sorting step, a recycling step such as pyrolysis, a cracking step such as steam cracking, a separation step to separate intermediates of one process step and further processing steps to convert such intermediates to output material(s), in particular cosmetic ingredient(s), 104 leaving the system boundary of the chemical production network 102.

The production operating system 106 of the chemical production network 102 may be configured to monitor and/or control the chemical production network 102 based on operating parameters of the different processes. One process step monitored and/or controlled may be the feed of input materials 100 or the release of output material(s), in particular cosmetic ingredient(s), 104. Another process step monitored and/or controlled may be the registration of environmental attributes associated with input materials 100 entering the system boundary of the chemical production network 102. Yet another process step monitored and/or controlled may be the attribution of environmental attributes to output material(s), in particular cosmetic ingredient(s), 104 produced via the chemical production network 102. Yet another process step monitored and/or controlled may be the management of environmental attributes associated with input materials 100 and output material(s), in particular cosmetic ingredient(s), 104 of the chemical production network 102.

The production operating system 106 may be configured to register inbound environmental attributes and to assign outbound environmental attributes. The production operating system 106 may be configured to access data related the inputs materials 100, the processes and/or the output material(s), in particular cosmetic ingredient(s), 104 used in the chemical production network 102. For example, the production operating system 106 may be configured to register a recycled or bio-based content of the one or more input material(s) 100 used in the chemical production network 102 as environmental attribute. The production operating system 106 may be configured to allocate the environmental attribute to at least one balancing account associated with the recycled or bio-based content of the input materials 100. The production operating system 106 may be configured to allocate at least a part of the environmental attributes from the at least one balancing account to the at least one output material(s), in particular to the cosmetic ingredient(s), 104.

The production operating system 102 may be configured to handle environmental attributes related to the input materials 100 and output material(s), in particular cosmetic ingredient(s), 104 of the chemical production network 102. For example, the production operating system 106 may be configured to determine environmental attributes associated with the use of input materials 100 impacting the environmental property of the chemical production network 102 and the output material(s), in particular cosmetic ingredient(s) 104 produced by the chemical production network 102. Further in particular, the production operating system 102 may be configured to determine environmental attributes associated with the output material(s), in particular cosmetic ingredient(s) 104. This way the production operating system 102 may be configured to store environmental attributes in balancing accounts or to withdraw environmental attributes from the balancing accounts. The environmental attributes may hence be viewed as a credit that may be deposited in an account or deducted from an account related to the input and output material(s), in particular cosmetic ingredient(s), of the chemical production network 102. This way the environmental impact of the production may be tracked and/or traced.

In chemical production networks 102 multiple value chains may be linked. Additionally different input materials 100 or chemical processes impacting the environmental property of output material(s), in particular cosmetic ingredient(s), 104 produced by the chemical production network 102 may be used. Examples of input materials 100 impacting at least one environmental property of output material(s), in particular cosmetic ingredient(s) 104 produced from such input materials 100 are recycled, renewable or bio-based input materials 104. Examples of chemical processes impacting the environmental property include chemical processes using environmentally friendly technology such as carbon capture, carbon utilization or heat pumps.

Owing to the processing of chemicals in continuous or semi-continuous production and the complexity of chemical production networks 102, traceability of the input materials through the network may be hampered. In such scenarios, an equivalent environmental attribute signifying the impact on the environmental property of output material(s), in particular cosmetic ingredient(s) 104 produced by the chemical production network may be allocated to balancing accounts and assigned to one or more output material(s), in particular cosmetic ingredient(s), 104 of the chemical production network 102. The environmental attributes may hence be decoupled from the physical material flow inside the chemical production network 102. Decoupling may be based on the mass balance model in that the equivalent amount assigned to the one or more output material(s), in particular cosmetic ingredient(s) may not exceed the equivalent amount provided by input materials or processes. If an equivalent amount has been allocated to the virtual account of one environmental attribute type, it may not be allocated a second time to another virtual account of the one environmental attribute type. Environmental attribute types may be recycled, bio-based, renewable or the like. Environmental attributes may be provided in the form of digital assets or cosmetic ingredient passports attached to the physical entity of the cosmetic ingredient.

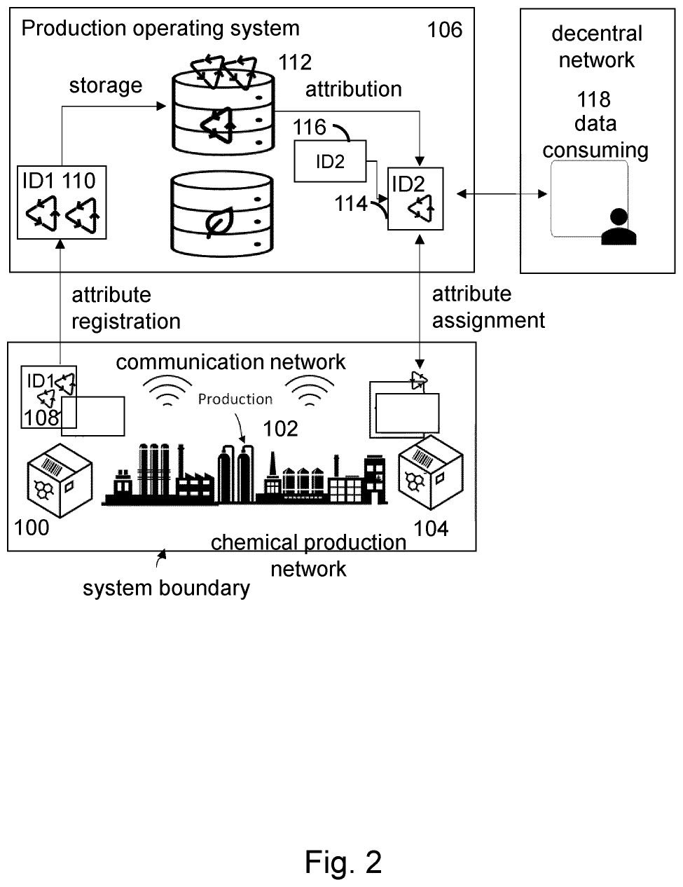

FIG. 2 illustrates schematically an example of attributing environmental attributes associated with input materials 100 to output material(s), in particular cosmetic ingredient(s) 104 of the chemical production network 102.

As shown in FIG. 1 the chemical production network 102 and operations of the chemical production network 102 may be monitored and/or controlled by a production operating system 106. The production operating system 106 may be configured to track environmental attributes from input materials 100 fed to the chemical production network 102 to output material(s), in particular cosmetic ingredient(s), 104 produced by the chemical production network 102. For tracking the operating system 106 may be configured to register environmental attributes associated with the input materials 100 provided to the chemical production network 102 and to attribute environmental attributes to output material(s), in particular cosmetic ingredient(s), 104 produced by the chemical production network 102.

The input materials 100 such as pyrolysis oil, bio-naphtha or bio-gas may be provided to the chemical production network 102. The input materials 100 may enter the chemical production network 104 at the entry point, such as a such as a steam cracker or a syngas plant. The input materials 100 may be used in the chemical production network 102 to produce one or more output material(s), in particular cosmetic ingredient(s), 104 from the input materials 100. output material(s), in particular cosmetic ingredient(s), 104 may be provided at exit points of the chemical production network 102. Further output material(s) may be MDI, TDI, PA6, EPS, PC, Polyols, Caprolactam, adipic acid, HMD, Polyamides.

On entry of the input material 100, input material data 108 may be provided via a communication network to a computing interface of the production operating system 106. A data provider, such as a QR code reader, may be configured to provide material data 108 related to the one or more input material(s) 100 and respective environmental attributes 108 to a computing interface configured to allocate the environmental attributes associated with the input materials 100. The material data 108 may include the input material identifier and environmental attributes associated with the input materials 100. The input material identifier may be associated with the physical entity of the input material 100 entering the chemical production network 102. The material data may be provided on, prior or after providing of the one or more input material(s) at entry points to the chemical production network 102.

The input material identifier may be linked to the environmental attribute(s) associated with the respective input material(s) 100, the amount of input material 100 and the certificate certifying the environmental attribute(s). The amount of input material may be a measured amount of input material 100 fed to a plant or storage of the chemical production network 102 for producing one or more output material(s), in particular cosmetic ingredient(s) 104 from the input material(s) 100. The input material identifier associated with the respective input material 100, the environmental attribute(s) associated with the respective input material(s) 100 and the amount of input material(s) 100 provided to the chemical production network 102 may be provided to the production operating system 106. Such data may be provided via a communication network on entry to chemical production network 102, or the data may be transferred from a computing system to the production operating system 106.

An inbound allocator 110 may be configured to allocate the one or more environmental attribute(s) to at least one balancing account 112 associated with the respective environmental attribute. For example, one balancing account 112 may relate to environmental attributes from recycled material and another balancing account 112 may relate to environmental attributes from bio-based material. The balancing account may be associated with the respective environmental attribute type, such as bio-based or recycled. Based on such association the balancing account associated with the environmental attribute type of the respective input material 100 may be selected. The environmental attributes may be allocated to the selected balancing account. For example, the account 112 for recycled material may be selected and the environmental attribute may be allocated to such account 122.

To allocate, the one or more environmental attribute(s) may be converted to balancing units and the balancing units may be allocated to the balancing account 122. The conversion may be based on a conversion factor such as mass, weight, carbon atoms, hydrogen atoms, methane equivalents or any other suitable measure for quantifying the environmental impact of the environmental attribute. The conversion factor may hence take into account the difference between producing chemical products from conventional input material(s) and producing chemical products from non-conventional input material(s) or producing chemical products from a mix of conventional and non-conventional input materials. The conversion factor may relate to differences in chemical and/or physical properties of conventional and non-conventional input material(s).

By using the balancing accounts 112 it can be ensured that environmental attributes of input materials 100 are only used once for assignment to cosmetic ingredients 104. This way double counting on input or output is avoided to ensure positive environmental impact can be reliable tracked and assigned to cosmetic ingredients 104.

An identifier provider 116 may be configured to provide the cosmetic ingredient identifier associated with the cosmetic ingredient produced by the chemical production network 102 and provided at the exit point from the chemical production network 102.

An outbound assigner 114 may be configured to assign at least one environmental attribute from the at least one balancing account 112 associated with the respective environmental attribute to the cosmetic ingredient identifier ID2. One or more environmental attribute(s) may be assigned to the at least one cosmetic ingredient identifier ID2. Assignment may include deallocation of the one or more environmental attributes from the balancing account 112 associated with the respective environmental attribute type. Assignment may include converting one or more balancing unit(s) to one or more environmental attribute(s).

Assigning at least one environmental attribute associated with input material(s) to cosmetic ingredient(s) may include the linking of the cosmetic ingredients identifier ID2 with the environmental attribute. The cosmetic ingredient identifier ID2 may be associated with the physical entity of the cosmetic ingredient. This way the virtual identifier of a material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

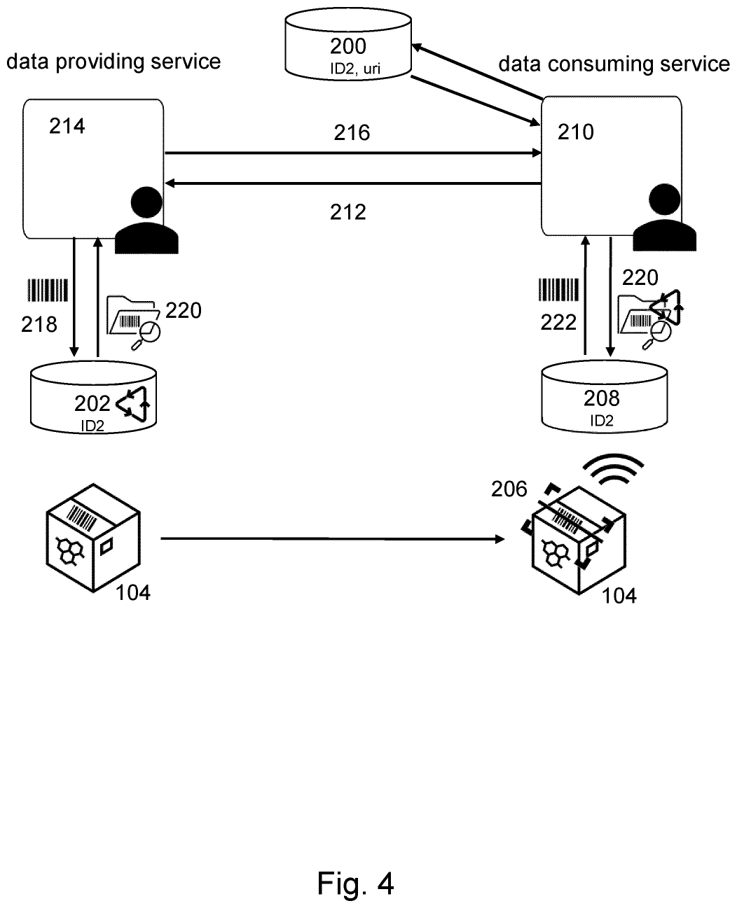

The outbound assigner 114 may be configured to provide the environmental attributes associated with the cosmetic ingredient to a data consumer, such as a system associated with a user of the cosmetic ingredient. The outbound assigner 114 may be configured to provide the environmental attributes associated with the cosmetic ingredient to a decentral network as will be described in the example of FIG. 4. Environmental attributes may be provided via the above ID based schema in the form of digital assets or cosmetic ingredient passports associated with the physical entity of the cosmetic ingredient.

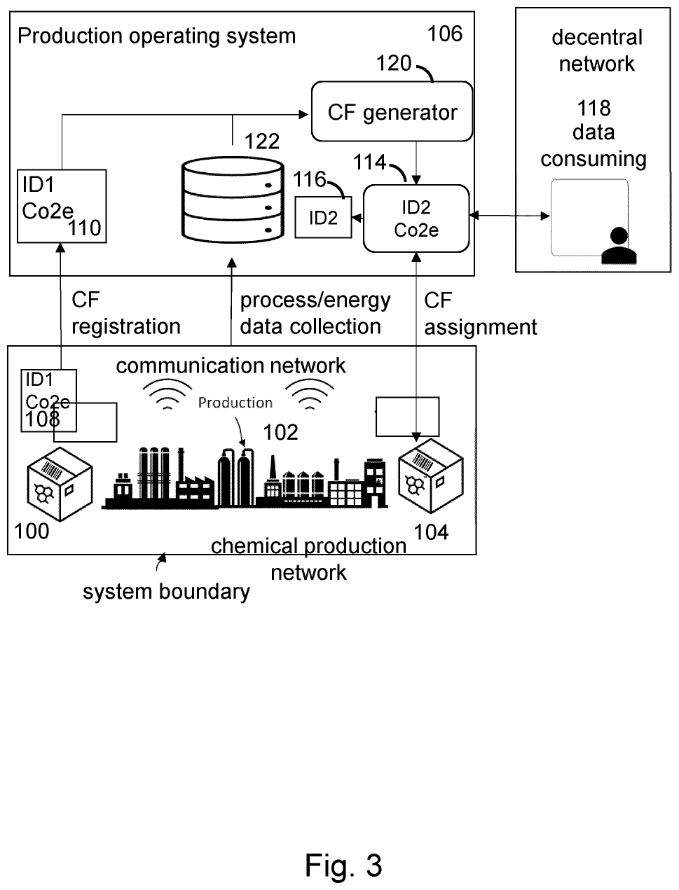

FIG. 3 illustrates schematically an example of attributing environmental attributes of input materials 100 and chemical processes to the cosmetic ingredient 104 of the chemical production network 102.

As described in the context of FIGS. 1 and 2 the chemical production network 102 and operations of the chemical production network 102 may be monitored and/or controlled by a production operating system 106. Input materials 100 such as pyrolysis oil, bio-naphtha or bio-gas may be provided to the chemical production network 102. The input materials 100 may be used in the chemical production network 102 to produce one or more cosmetic ingredient(s) 104 from the input materials 100.

On entry of the input material 100, input material data 108 may be provided via a communication network to a computing interface of the production operating system 106. A data provider, such as a QR code reader, may be configured to provide material data 108 related to the one or more input material(s) 100 and respective environmental attributes 108 to a computing interface configured to allocate the environmental attributes associated with the input materials 100. The material data 108 may include the input material identifier and environmental attributes associated with the input materials 100. The input material identifier may be associated with the physical entity of the input material 100 entering the chemical production network 102. The input material identifier may be linked to the carbon footprint of the input material 100 as environmental attribute. The material data may be provided on, prior or after providing of the one or more input material(s) at entry points to the chemical production network 102.

The inbound allocator 110 may be configured to retrieve the one or more environmental attribute(s) and to provide such attributes to the carbon footprint (CF) generator 120. A process data provider 122 may be configured to gather process data associated with the chemical processing of the input material(s) 100 to produce the cosmetic ingredient(s) 104. The process data provider 122 may be configured to gather energy data associated with the energy consumption of the chemical processing. The process data provider 122 may be configured to provide the process data and the energy data to the CF generator 120.

The CF generator 120 may be configured to determine the carbon footprint of the cosmetic ingredient produced by the chemical production network. The carbon footprint of the of the cosmetic ingredient may be determined based on the process data, the energy data and the carbon footprint of the input material(s) 100 used to produce the cosmetic ingredient.

An identifier provider 116 may be configured to provide the cosmetic ingredient identifier associated with the cosmetic ingredient produced by the chemical production network 102 and provided at the exit point from the chemical production network 102.

An outbound assigner 114 may be configured to assign the determined carbon footprint to the cosmetic ingredient identifier ID2. One or more environmental attribute(s) may be assigned to the at least one cosmetic ingredient identifier ID2, such as described in the context of FIG. 2.

The outbound assigner 114 may be configured to provide the environmental attributes, in particular the carbon footprint, associated with the cosmetic ingredient to a data consumer, such as a system associated with a user of the cosmetic ingredient. The outbound assigner 114 may be configured to provide the environmental attributes associated with the cosmetic ingredient to a decentral network as will be described in the example of FIG. 4. Environmental attributes may be provided via the above ID based schema in the form of digital assets or cosmetic ingredient passports associated with the physical entity of the cosmetic ingredient.

FIG. 4 illustrates schematically an example of a method or apparatus for providing environmental attributes associated with cosmetic ingredients to a material user as data consumer via a decentral network.

The cosmetic ingredient 104 as produced by the chemical production network 102 may be provided in association with the digital asset as described in the context of FIGS. 2 and 3. The digital asset may include the cosmetic ingredient identifier. The digital asset may include one or more environmental attribute(s) such as the product carbon footprint, recycled content or bio-based content. The digital asset may relate to one or more environmental attribute(s) such as the product carbon footprint, recycled content or bio-based content. The digital asset may include a digital representation of one or more environmental attribute(s) such as the product carbon footprint, recycled content or bio-based content.

The digital asset may further include or relate to authentication and/or authorization information linked to the cosmetic ingredient identifier. The authentication and/or authorization information may be provided for authentication and/or authorization of a data providing service 208 and/or data consuming service 210. The cosmetic ingredient identifier may include or relate to a decentral identifier, that is uniquely associated with the cosmetic ingredient. The decentral identifier may be connected to the digital representation of the environmental attributes. The digital representation may include a representation for accessing the environmental attributes or parts thereof. The decentral identifier may include a Universally Unique IDentifier (UUID) or a Digital IDentifier (DID). The decentral identifier may include any unique identifier uniquely associated with a data owner and/or cosmetic ingredient. The data owner may be the producer of the cosmetic ingredient. Via the decentral identifier and its unique association with the data owner and/or cosmetic ingredient access to the material configuration data may be controlled by the data owner.

The digital asset including the digital representation of one or more environmental attribute(s) such as the product carbon footprint, recycled content or bio-based content may be stored in a decentral data base 200. The one or more environmental attribute(s) such as the product carbon footprint, recycled content or bio-based content may be stored in a data base 202 associated with the data owner, such as the producer of the cosmetic ingredient 104.

The cosmetic ingredient 104 may be physically delivered to a user of the cosmetic ingredient. The cosmetic ingredient may be connected with a QR-code having encoded the cosmetic ingredient identifier. The user of the cosmetic ingredient may read the QR-code through a QR-code reader 206. The cosmetic ingredient identifier may be provided to a data base 208 associated with the consumer of the cosmetic ingredient 104. In other embodiments the consumer of the cosmetic ingredient may retrieve the cosmetic ingredient identifier through the decentral data base 200.

The data owner in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service 210 may comprise computer-executable instructions for accessing and/or processing data. The data providing service 210 may comprise computer-executable instructions for providing and/or processing data, such as cosmetic ingredient data, associated with the data owner for accessing and/or processing by the data consuming service 214.

Based on the received cosmetic ingredient identifier a request to access the environmental attributes associated with the cosmetic ingredient identifier may be triggered by the data consuming service 210 as signified by arrow 212. The cosmetic ingredient identifier may be provided to the data providing service 214 associated with or of the producer of the cosmetic ingredient 104. In addition, authentication and/or authorization information may be provided.

The request may be authenticated and/or authorized to access the environmental attributes associated with the cosmetic ingredient identifier. Based on successful authorization and/or authentication access to the environmental attributes associated with the cosmetic ingredient identifier may be granted.

For access the cosmetic ingredient identifier may be provided to the data providing service 214 as signified by arrow 212. The data providing service 214 may use the received cosmetic ingredient identifier to retrieve the environmental attributes associated with the cosmetic ingredient 104 as signified by arrows 218 and 220. The environmental attributes associated with the cosmetic ingredient 104 provided to the data providing service 214 may be provided to the data consuming service 210 as signified by arrow 216. The environmental attributes associated with the cosmetic ingredient 104 may be stored in the data base 208 associated with the user of the cosmetic ingredient 104 as signified by arrow 220.

Through the output identifier or decentral identifier, the environmental attributes can be uniquely associated with the cosmetic ingredient. Through the decentral network the environmental attributes may be transferred between the producer of the cosmetic ingredient and the user of the cosmetic ingredient. This way the environmental attributes can be shared with unique association to the cosmetic ingredient and without central intermediary directly between the value chain players. This allows for transparency of environmental attributes across the value chain and positive environmental impacts from cosmetic ingredients produced by the chemical production network 102 can be tracked through the value chain.

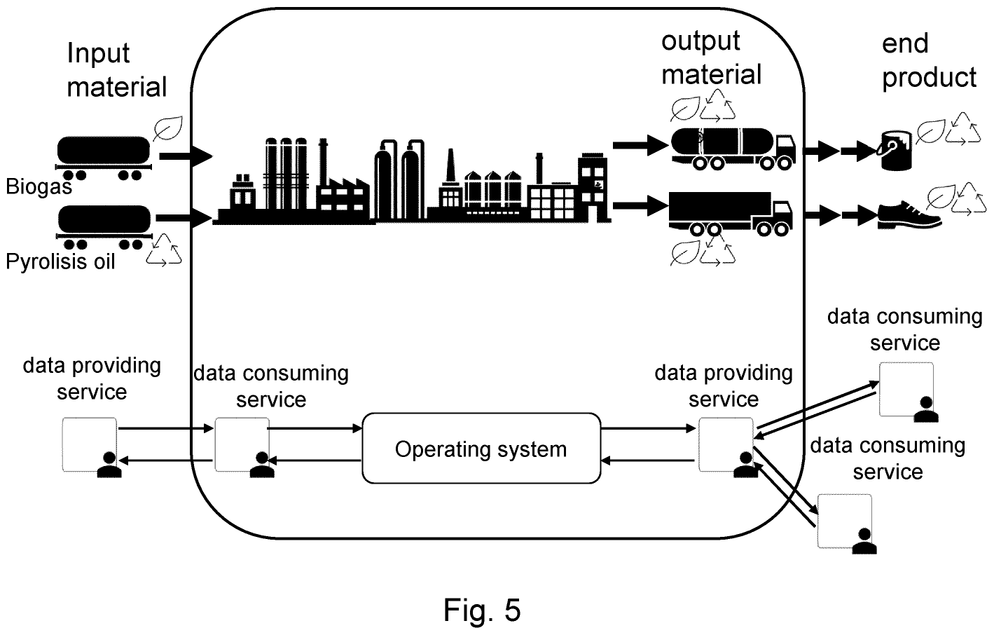

FIG. 5 illustrates schematically an example of a method or apparatus for providing environmental attributes associated with cosmetic ingredients across value chains via the decentral network.

In the example of FIG. 5 a fully connected value chain including the chemical production network is illustrated. In the example, the input material provider, the cosmetic ingredient producer, the cosmetic ingredient user and the end product producer may be connected to the decentral network as described in the context of FIG. 4. Environmental attributes may be provided via the ID based schema described in the context of FIGS. 2-4 in the form of digital assets or cosmetic ingredient passports associated with the physical entity of the input material, the cosmetic ingredient, any intermediate product or the end product.

The input material provider may provide the input materials such as bio-gas or pyrolysis oil. The environmental attributes of the input material may be provided through the data providing service connected to the decentral network as described in the context of FIG. 4. The cosmetic ingredient producer may produce the cosmetic ingredient from the input material(s) provided to the chemical production network. The cosmetic ingredient producer may access the environmental attributes associated with the input material through a data consuming service connected to the decentral network as described in the context of FIG. 4. The cosmetic ingredient producer may manage the environmental attributes via the production operating system as described in the context of FIGS. 1 to 3. The cosmetic ingredient producer may assign the environmental attributes associated with the input materials or environmental attributes associated with the chemical production network such as the carbon footprint, to the cosmetic ingredients as described in the context of FIGS. 1 to 3. The cosmetic ingredient producer may provide the environmental attributes associated with the cosmetic ingredient through the data providing service connected to the decentral network as described in the context of FIG. 4. The cosmetic ingredient user or the end product producer may access the environmental attributes associated with the cosmetic ingredient through the data consuming service connected to the decentral network as described in the context of FIG. 4.

The respective data owners in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service may comprise computer-executable instructions for accessing and/or processing data, such as cosmetic ingredient data, associated with the data owner. The data providing service may comprise computer-executable instructions for providing and/or processing data, such as cosmetic ingredient data, associated with the data owner for accessing and/or processing by the data consuming service.

In the example of FIG. 5 the decentral identifier may relate to the end product. Such decentral identifier may be provided to the value chain participants. Via the end product specific decentral identifier data associated with the end product produced from the cosmetic ingredient may be gathered across the production chain and assigned to the end product specific decentral identifier. For example, the one or more environmental attribute(s) associated with the end product may be derived from the environmental attribute(s) associated with the cosmetic ingredient, the input material or any other product entity present in the value chain of the end product.

This way the environmental attributes of input materials, cosmetic ingredients and any products produced from cosmetic ingredients may be tracked through the value chain up to the end product. By tracking the environmental attributes of materials in such way the information can be made transparent across the value chain while the information flow can be controlled by the participants in the supply chain. In addition, the environmental attributes can be handled according to the individual participants needs by production operating systems as described in the context of FIGS. 1 to 3. Overall, such tracking enables tracking of positive environmental impact by individual supply chain participants, which makes positive environmental impacts transparent and attributable to individual supply chain participants.

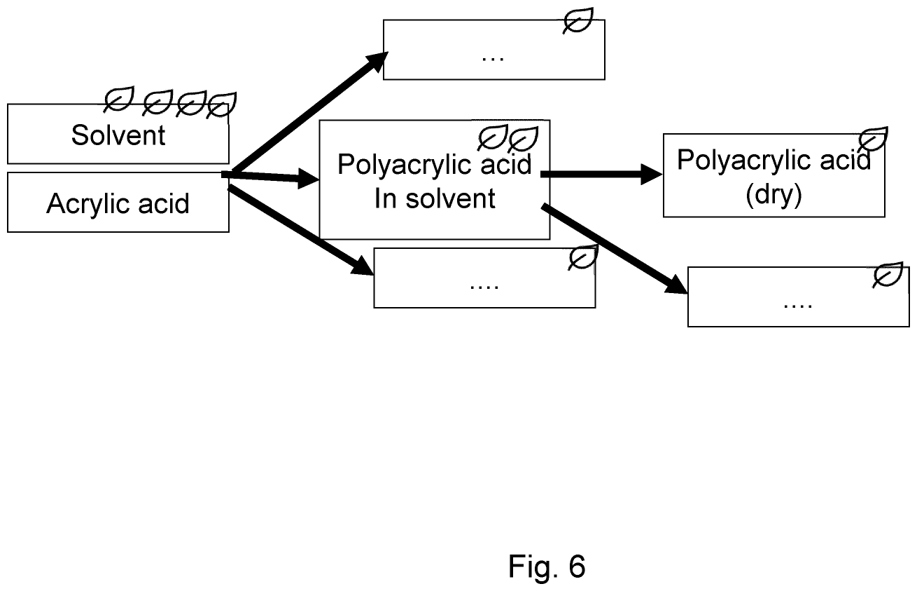

FIG. 6 illustrates schematically an example of a chemical production network for producing polyacrylic acid associated with the digital asset. The example of FIG. 6 is an illustrative example and should not be considered limiting.

In the example of FIG. 6 acrylic acid may be provided to the chemical production network for producing as an example polyacrylic acid. Acrylic acid may be the input material in this example as mixed feedstock. From acrylic acid polyacrylic acid and other products, such as intermediate materials or output materials, may be produced.

In the example of FIG. 6 the Greenhouse Gas emissions as non-fossil input material may be tracked. As described in the context of FIG. 2, the Greenhouse Gas emissions at the start of the polyacrylic acid production chain may be registered via the production operating system 106. Here the Greenhouse Gas emissions may be allocated to the balancing account 112 for acrylic acid. The environmental attribute of acrylic acid is hence detached from the mass flows of the chemical processing in the chemical production network 102 as illustrated in FIG. 6.

For attribution of the acrylic acid used to produce the polyacrylic acid, the Greenhouse Gas emissions may be determined. The Greenhouse Gas emissions attributable to polyacrylic acid production may be based on mass conservation attributable to the produced polyacrylic acid.

For example, only half of the Greenhouse Gas emissions may be attributable to the polyacrylic acid and the other half may be attributable to other output products resulting from the acrylic acid as input material. The environmental attribute Greenhouse Gas emissions may be attributed to such extend to the polyacrylic acid.

In the system shown in FIG. 2, the environmental attribute associated with the production of polyacrylic acid may be attributed to the produced polyacrylic acid by associating the decentral ID to the polyacrylic acid and assigning the environmental attribute to the decentral ID. By linking the ID and the environmental attribute, the environmental attribute may be uniquely linked to the produced polyacrylic acid. The polyacrylic acid may be delivered to the polyacrylic acid user. The packaging such as lose bags with polyacrylic acid may include a QR-code. The decentral ID may be included or encoded to the QR code. This way polyacrylic acid user may access the environmental attributes associated to the polyacrylic acid via the ID based protocol described in the context of FIGS. 4 and 5.

Similarly to this example the chemical production network for producing polyacrylic acid associated with the digital asset may be based on the method illustrated in FIG. 3 for carbon footprints.

Similarly to this example the registration of carbon footprint data associated with input materials and production processes as well as the attribution of said carbon footprint to the produced functional group compound composition may be based on the method illustrated in FIG. 3 for carbon footprints.

The present disclosure has been described in conjunction with preferred embodiments and examples as well. However, other variations can be understood and effected by those persons skilled in the art and practicing the claimed invention, from the studies of the drawings, this disclosure and the claims.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

1. A system for producing a cosmetic ingredient associated with a digital asset, the system comprising:
a chemical production network configured to produce the cosmetic ingredient wherein the cosmetic ingredient is produced from one or more input material through one or more chemical process of the chemical production network, wherein the one or more input material and/or the one or more chemical process are associated with one or more environmental attribute;
a production operating apparatus configured to generate the digital asset by providing a decentral identifier associated with the produced cosmetic ingredient and one or more environmental attribute of the one or more input material and/or the one or more chemical process;
linking the decentral identifier and the one or more environmental attribute;
a chemical production network or system configured to provide the produced cosmetic ingredient in association with the digital asset.

2. The system of claim 1, wherein the cosmetic ingredient associated with the digital asset is selected from the group consisting of an emollient, a surfactant, a rheology modifier, a stabilizer, a solvent, a solubilizer, a preservative, a neutralizing agent, a buffer, a complexing agent, an active biogenic ingredient, an antioxidant, a UV light protection filter, a self-tanning agent, a humectant, an insect repellent, a tyrosine inhibitor, a coolant, a perfume oil, a film former, a dye, an effect pigment, and a sensory additive.

3. The system of claim 1, wherein the digital asset of the cosmetic ingredient includes mass balanced environmental attributes related to the one or more input material.

4. The system of claim 1, wherein the one or more environmental attribute associated with the cosmetic ingredient is provided from at least one balancing account configured to store the one or more environmental attribute associated with the one or more input material.

5. The system of claim 1, wherein the one or more environmental attribute is associated with at least one property related to the environmental impact of the one or more input material and/or the one or more chemical process.

6. The system of claim 1, wherein the production operating apparatus is configured to gather one or more environmental attribute associated with the produced cosmetic ingredient before, during, and/or after production of the cosmetic ingredient by the chemical production network.

7. The system of claim 1, wherein the one or more environmental attribute associated with the cosmetic ingredient produced through the one or more chemical from the one or more input provided to the chemical production network include the one or more environmental attribute associated with the one or more input material, the one or more chemical process and/or the chemical production network.

8. The system of claim 1, wherein the one or more environmental attribute associated with the one or more input material is provided before, during, and/or after production of the cosmetic ingredient by the chemical production network, wherein the one or more environmental attribute associated with the one or more input material is allocated to at least one balancing account before, during, and/or after production of the cosmetic ingredient by the chemical production network.

9. The system of claim 1, wherein the one or more environmental attribute associated with the produced cosmetic ingredient relate to an environmental property generated from process data associated with the chemical processing of the one or more input material and/or energy data associated with the energy consumption of the chemical processing, wherein the one or more environmental attribute associated with the produced cosmetic ingredient are generated before, during, and/or after production of the cosmetic ingredient by the chemical production network.

10. A method for producing a cosmetic ingredient associated with a digital asset, wherein the method comprises:
producing the cosmetic ingredient from one or more input material-through one or more chemical process of a chemical production network, wherein the one or more input material and/or the one or more chemical process is associated with the one or more environmental attribute;
generating the digital asset by
providing a decentral identifier associated with the produced cosmetic ingredient and the one or more environmental attribute associated with the one or more input material and/or the one or more chemical process;
linking the decentral identifier and the one or more environmental attribute;
providing the produced cosmetic ingredient in association with the digital asset.

11. A cosmetic ingredient associated with a digital asset including a decentral identifier associated with the cosmetic ingredient and linked to one or more environmental attribute of the one or more input material and/or the one or more chemical process used to produce the cosmetic ingredient.

12. The cosmetic ingredient of claim 11, wherein the cosmetic ingredient is selected from the group consisting of an emollient, a surfactant, a rheology modifier, a stabilizer, a solvent, a solubilizer, a preservative, a neutralizing agent, a buffer, a complexing agent, an active biogenic ingredient, an antioxidant, a UV light protection filter, a self-tanning agent, a humectant, an insect repellent, a tyrosine inhibitor, a coolant, a perfume oil, a film former, a dye, an effect pigment, and a sensory additive.

13. A method for generating a digital asset associated with a cosmetic ingredient, wherein the cosmetic ingredient is produced from one or more input material through one or more chemical process of a chemical production network, wherein the one or more input material and/or the one or more chemical process are associated with the one or more environmental attribute, the method comprising:
providing a decentral identifier associated with the produced cosmetic ingredient and the one or more environmental attribute of the one or more input material and/or the one or more chemical process used to produce the cosmetic ingredient;
linking the decentral identifier and the one or more environmental attribute;
providing the digital asset in association with the produced cosmetic ingredient, wherein the one or more environmental attribute associated with the cosmetic ingredient is made accessible to a user of the cosmetic ingredient through the digital asset.

14. A digital asset as generated according to the method of claim 13.

15. A method for using the digital asset generated according to the method of claim 13 in production of a product produced from the cosmetic ingredient associated with the digital asset.

16. A method for using the cosmetic ingredient of any of claim 11 associated with the digital asset for producing a product from the cosmetic ingredient and deriving a digital asset associated with the product from the cosmetic ingredient digital asset.
